(19) United States
(12) Patent Application Publication
(10) Pub. No.: US 2021/0395650 A1
(43) Pub. Date: Dec. 23, 2021

(54) COMPOUNDS STABILIZING HYDROLASES IN LIQUIDS

(71) Applicant: BASF SE, Ludwigshafen am Rhein (DE)

(72) Inventors: Stephan Hueffer, Ludwigshafen (DE); OLiver Spangenberg, Ludwigshafen (DE); Grit Baier, Ludwigshafen (DE); Alejandra Garcia Marcos, Ludwigshafen (DE); Claudia Esper, Ludwigshafen (DE)

(21) Appl. No.: 17/281,496

(22) PCT Filed: Sep. 24, 2019
(86) PCT No.: PCT/EP2019/075672

§ 371 Date: Mar. 30, 2021

(30) Foreign Application Priority Data
Oct. 5, 2018 (EP) 18198955.9
Oct. 10, 2018 (EP) 18199687.7
Oct. 10, 2018 (EP) 18199698.4

(51) Int. Cl. C11D 3/386; C12N 9/96; C12N 9/64

(52) U.S. Cl. CPC C11D 3/38663; C11D 3/38618; C12Y 304/21062; C12N 9/6424; C12N 9/96

(57) ABSTRACT

Described herein is an enzyme preparation including
component (a): at least one compound according to general formula (I)

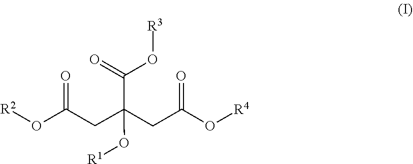

wherein
R¹ is;
R², R³, R⁴ are independently from each other selected from the group consisting of H, linear C₁-C₈ alkyl, and branched C₃-C₈ alkyl, C₆-C₁₀-aryl, non-substituted or substituted with one or more carboxylate or hydroxyl groups, and C₆-C₁₀-aryl-alkyl, wherein an alkyl of the C₆-C₁₀-aryl-alkyl is selected from the group consisting of linear C₁-C₈ alkyl and branched C₃-C₈ alkyl, wherein at least one of R², R³, and R⁴ is not H,
component (b): at least one enzyme selected from the group consisting of hydrolases (EC 3);
and optionally
component (c): at least one compound selected from the group consisting of solvents, enzyme stabilizers different from component (a), and compounds stabilizing the enzyme preparation.

The present invention is directed towards an enzyme preparation, preferably a liquid enzyme preparation, comprising
component (a): at least one compound according to general formula (I)

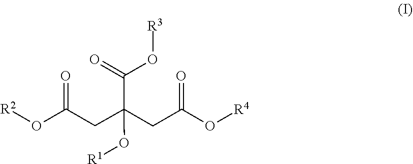

wherein the variables in formula (I) are as follows:
R¹ is selected from H and C₁-C₁₀ alkylcarbonyl, wherein alkyl may be linear or branched and may bear one or more hydroxyl groups,
R², R³, R⁴ are independently from each other selected from H, linear C₁-C₅ alkyl, and branched C₃-C₁₀ alkyl, C₆-C₁₀-aryl, non-substituted or substituted with one or more carboxylate or hydroxyl groups, and C₆-C₁₀-aryl-alkyl, wherein alkyl of the latter is selected from linear C₁-C₈ alkyl or branched C₃-C₈ alkyl, wherein at least one of R², R³, and R⁴ is not H;
component (b): at least one enzyme selected from the group of hydrolases (EC 3), preferably at least one enzyme selected from the group of proteases, more preferably at least one enzyme selected from the group of serine endopeptidases (EC 3.4.21), most preferably at least one enzyme selected from the group of subtilisin type proteases (EC 3.4.21.62);
and optionally
component (c): at least one compound selected from solvents, enzyme stabilizers different from component (a), and compounds stabilizing the liquid enzyme preparation as such.

Enzymes are usually produced commercially as a liquid concentrate, frequently derived from a fermentation broth. The enzyme tends to loose enzymatic activity if it remains in an aqueous environment and so it is conventional practice to convert it to an anhydrous form: aqueous concentrates may be lyophilized or spray-dried e.g. in the presence of a carrier material to form aggregates. Usually, solid enzyme products need to be “dissolved” prior to use. To stabilize enzymes in liquid products enzyme inhibitors are usually employed, preferably reversible enzyme inhibitors, to inhibit enzyme activity temporarily until the enzyme inhibitor is released. Boric acid and boronic acids are known to reversibly inhibit proteolytic enzymes. A discussion of the inhibition of one serine protease, subtilisin, by boronic acid is provided in Molecular & Cellular Biochemistry 51, 1983, pp. 5-32. For reactivation, this inhibitor needs to be removed prior or during application, which can be done for example by dilution.

Because of environmental considerations there is a demand for at least reducing the amounts of boron-containing compounds used for enzyme stabilization. There is a seek for alternatives to be used as enzyme stabilizers in the presence of enzymes.

The problem to be solved for the current invention relates to providing a compound helping to reduce loss of enzymatic activity during storage of liquid enzyme containing products. It was a further objective of the present invention to provide an enzyme preparation that allows to be flexibly formulated into liquid detergent formulations or cleaning formulations with either one type of enzymes or mixtures of enzymes.

The problem was solved by providing compounds according to general formula (I):

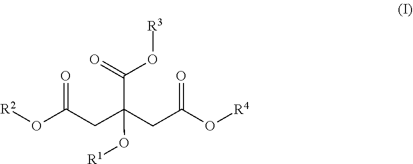

wherein the variables in formula (I) are as follows:

R¹ is selected from H and C₁-C₁₀ alkylcarbonyl, wherein alkyl may be linear or branched and may bear one or more hydroxyl groups,

R², R³, R⁴ are independently from each other selected from H, linear C₁-C₅ alkyl, and branched C₃-C₁₀ alkyl, C₆-C₁₀-aryl, non-substituted or substituted with one or more carboxylate or hydroxyl groups, and C₆-C₁₀-aryl-alkyl, wherein alkyl of the latter is selected from linear C₁-C₈ alkyl or branched C₃-C₈ alkyl, wherein at least one of R², R³, and R⁴ is not H; and

wherein said compound supports retention of enzymatic activity of at least one enzyme selected from the group of hydrolases (EC 3), preferably from the group of proteases, more preferably from serine endopeptidases (EC 3.4.21), most preferably from the group of subtilisin type proteases (EC 3.4.21.62); during storage of the same within liquid products.

Enzyme names are known to those skilled in the art based on the recommendations of the Nomenclature Committee of the International Union of Biochemistry and Molecular Biology (IUBMB). Enzyme names include: an EC (Enzyme Commission) number, recommended name, alternative names (if any), catalytic activity, and other factors; see http://www.sbcs.qmul.ac.uk/iubmb/enzyme/EC3/ in the version last updated on 28 Jun. 2018. In one aspect, the invention provides an enzyme preparation containing
component (a): at least one enzyme stabilizer selected from compounds according to general

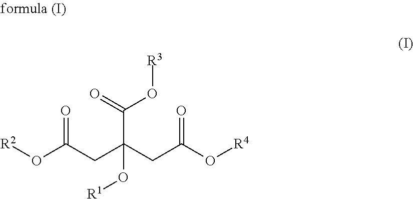

wherein the variables in formula (I) are as follows:
R¹ is selected from H and C₁-C₁₀ alkylcarbonyl, wherein alkyl may be linear or branched and may bear one or more hydroxyl groups,
R², R³, R⁴ are independently from each other selected from H, linear C₁-C₅ alkyl, and branched C₃-C₁₀ alkyl, C₆-C₁₀-aryl, non-substituted or substituted with one or more carboxylate or hydroxyl groups, and C₆-C₁₀-aryl-alkyl, wherein alkyl of the latter is selected from linear C₁-C₈ alkyl or branched C₃-C₈ alkyl, wherein at least one of R², R³, and R⁴ is not H, and
component (b): at least one enzyme selected from the group of hydrolases (EC 3), preferably at least one enzyme selected from the group of proteases, more preferably at least one enzyme selected from the group of serine endopeptidases (EC 3.4.21), most preferably at least one enzyme selected from the group of subtilisin type proteases (EC 3.4.21.62);
and optionally
component (c): at least one compound selected from solvents, enzyme stabilizers different from component (a), and compounds stabilizing the liquid enzyme preparation as such.

The enzyme preparation of the invention may be liquid at 20° C. and 101.3 kPa. Liquids include solutions, emulsions and dispersions, gels etc. as long as the liquid is fluid and pourable. In one embodiment of the present invention, liquid detergent compositions according to the present invention have a dynamic viscosity in the range of about 500 to about 20,000 mPa*s, determined at 25° C. according to Brookfield, for example spindle 3 at 20 rpm with a Brookfield viscosimeter LVT-II.

In one embodiment, liquid means that the enzyme preparation does not show visible precipitate formation or turbidity after storage of the liquid enzyme preparation, preferably after at least 20 days of storage at 37° C.

### Component (a)

More specifically, component (a) is a compound of general formula (I)

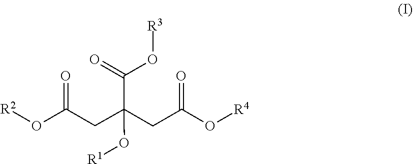

wherein the variables in formula (I) are defined as follows:

R¹ is selected from H and C₁-C₁₀ alkylcarbonyl, wherein alkyl may be linear or branched and may bear one or more hydroxyl groups,

R², R³, R⁴ are independently from each other selected from H, linear C₁-C₈ alkyl, and branched C₃-C₈ alkyl, C₆-C₁₀-aryl, non-substituted or substituted with one or more carboxylate or hydroxyl groups, and C₆-C₁₀-aryl-alkyl, wherein alkyl of the latter is selected from linear C₁-C₈ alkyl or branched C₃-C₈ alkyl, wherein at least one of R², R³, and R⁴ is not H. Examples of linear C₁-C₈ alkyl are methyl, ethyl, n-propyl, n-butyl, n-pentyl, etc. Examples of branched C₃-C₈ alkyl are 2-propyl, 2-butyl, sec.-butyl, tert.-butyl, 2-pentyl, 3-pentyl, iso-pentyl, etc. Examples of C₆-C₁₀-aryl, non-substituted or substituted with one or more carboxylate or hydroxyl groups, are phenyl, 1-naphthyl, 2-naphthyl, ortho-phenylcarboxylic acid group, meta-phenylcarboxylic acid group, para-phenylcarboxylic acid group, ortho-hydroxyphenyl, para-hydroxyphenyl, etc.

In one embodiment, R¹ in the compound according to formula (I) is selected from H, acetyl and propionyl. In one embodiment, R¹ in the compound according to formula (I) is H. In one embodiment, R¹ in the compound according to formula (I) is acetyl. In one embodiment, R¹ in the compound according to formula (I) is propionyl.

In one embodiment, R² in the compound according to formula (I) is H, and R³, R⁴ are independently from each other selected from linear C₁-C₈ alkyl, and branched C₃-C₈ alkyl, C₆-C₁₀-aryl, non-substituted or substituted with one or more carboxylate or hydroxyl groups, and C₆-C₁₀-aryl-alkyl, wherein alkyl of the latter is selected from linear C₁-C₈ alkyl or branched C₃-C₈ alkyl.

In one embodiment, R², R³, R⁴ in the compound according to formula (I) are the same, wherein R², R³, R⁴ are selected from linear C₁-C₈ alkyl, and branched C₃-C₈ alkyl, C₆-C₁₀-aryl, non-substituted or substituted with one or more carboxylate or hydroxyl groups, and C₆-C₁₀-aryl-alkyl, wherein alkyl of the latter is selected from linear C₁-C₈ alkyl or branched C₃-C₈ alkyl. In one embodiment, R¹ in the compound according to formula (I) is H, and R², R³, R⁴ are selected from linear C₂-C₄ alkyl, phenylmethyl, and ortho-phenylcarboxylic acid group (salicyl).

In one embodiment, R¹, R² and R³ in the compound according to formula (I) are H, and R⁴ is selected from linear C₂-C₄ alkyl, preferably C₂ alkyl. In one embodiment, R¹, and R² in the compound according to formula (I) are H, and R³ and R⁴ are selected from linear C₂-C₄ alkyl, preferably C₂ alkyl.

In one embodiment, R¹ in the compound according to formula (I) is acetyl, and R², R³, R⁴ are selected from linear C₂-C₄ alkyl, preferably 02 and 04 alkyl.

Component (a) includes salts of the compound according to formula (I). Salts include alkali metal and ammonium salts e.g those of mono- and triethanolamine. Preference is given to potassium salts and sodium salts.

In one embodiment of the present invention, enzyme preparations, preferably liquid enzyme preparations, comprise component (a) in amounts in the range of 0.1% to 30% by weight, relative to the total weight of the enzyme preparation. The enzyme preparation may comprise component (a) in amounts in the range of 0.1% to 15% by weight, 0.25% to 10% by weight, 0.5% to 10% by weight, 0.5% to 6% by weight, or 1% to 3% by weight, all relative to the total weight of the enzyme preparation.

In one embodiment of the present invention, compound (a) comprises at least one at least partially hydrolyzed derivative of compound (a) as impurity. In one embodiment of the present invention, component (a) comprises as an impurity of a fully hydrolyzed compound (a′) which is as follows:

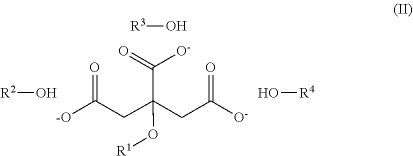

wherein the variables R¹, R², R³, and R⁴ are the same as described for component (a) above. Such impurity may amount to up to 50 mol-%, preferably 0.1 to 20 mol-%, even more preferably 1 to 10 mol-% of component (a). Although the impurities may originate from the synthesis of component (a) and may be removed by purification methods it is not preferred to remove it.

### Component (b)

In one aspect of the invention, at least one enzyme comprised in component (b) is part of a liquid enzyme concentrate. “Liquid enzyme concentrate” herein means any liquid enzyme-comprising product comprising at least one enzyme. “Liquid” in the context of enzyme concentrate is related to the physical appearance at 20° C. and 101.3 kPa.

The liquid enzyme concentrate may result from dissolution of solid enzyme in solvent. The solvent may be selected from water and an organic solvent. A liquid enzyme concentrate resulting from dissolution of solid enzyme in solvent may comprise amounts of enzyme up to the saturation concentration.

Dissolution herein means, that solid compounds are liquified by contact with at least one solvent. Dissolution means complete dissolution of a solid compound until the saturation concentration is achieved in a specified solvent, wherein no phase-separation occurs.

In one aspect of the invention, component (b) of the resulting enzyme concentrate may be free of water, meaning that no significant amounts of water are present. Non-significant amounts of water herein means, that the enzyme preparation comprises less than 25%, less than 20%, less than 15%, less than 10%, less than 7%, less than 5%, less than 4%, less than 3%, less than 2% by weight water, all relative to the total weight of the enzyme concentrate, or no water. In one embodiment, enzyme concentrate free of water free of water means that the enzyme concentrate does not comprise significant amounts of water but does comprise organic solvents in amounts of 30-80% by weight, relative to the total weight of the enzyme concentrate.

Liquid enzyme concentrates comprising water may be called “aqueous enzyme concentrates”. Aqueous enzyme concentrates may be enzyme-comprising solutions, wherein solid enzyme product has been dissolved in water. In one embodiment “aqueous enzyme concentrate” means enzyme-comprising products resulting from enzyme production by fermentation.

Fermentation means the process of cultivating recombinant cells which express the desired enzyme in a suitable nutrient medium allowing the recombinant host cells to grow (this process may be called fermentation) and express the desired protein. At the end of the fermentation, fermentation broth usually is collected and further processed, wherein the fermentation broth comprises a liquid fraction and a solid fraction. Depending on whether the enzyme has been secreted into the liquid fraction or not, the desired protein or enzyme may be recovered from the liquid fraction of the fermentation broth or from cell lysates. Recovery of the desired enzyme uses methods known to those skilled in the art. Suitable methods for recovery of proteins or enzymes from fermentation broth include but are not limited to collection, centrifugation, filtration, extraction, and precipitation.

Liquid enzyme concentrates, may comprise amounts of enzyme in the range of 0.1% to 40% by weight, or 0.5% to 30% by weight, or 1% to 25% by weight, or 3% to 25% by weight, or 5% to 25% by weight, all relative to the total weight of the enzyme concentrate. In one embodiment, liquid enzyme concentrates are resulting from fermentation and are aqueous.

Aqueous enzyme concentrates resulting from fermentation may comprise water in amounts of more than about 50% by weight, more than about 60% by weight, more than about 70% by weight, or more than about 80% by weight, all relative to the total weight of the enzyme concentrate. Aqueous enzyme concentrates which result from fermentation, may comprise residual components such as salts originating from the fermentation medium, cell debris originating from the production host cells, metabolites produced by the production host cells during fermentation. In one embodiment, residual components may be comprised in liquid enzyme concentrates in amounts less than 30% by weight, less than 20% by weight less, than 10% by weight, or less than 5% by weight, all relative to the total weight of the aqueous enzyme concentrate.

At least one enzyme comprised in component (b) is selected from hydrolases (EC 3), hereinafter also referred to as enzyme (component (b)). Preferred enzymes (component (b)) are selected from the group of enzymes acting on ester bond (E.C. 3.1), glycosylases (E.C. 3.2), and peptidases (E.C. 3.4). Enzymes acting on ester bond (E.C. 3.1), are hereinafter also referred to as lipases (component (b)), respectively. Glycosylases (E.C. 3.2) are hereinafter also referred to as either amylases (component (b)) and cellulases (component (b)). Peptidases are hereinafter also referred to as proteases (component (b)).

Hydrolases (component (b)) in the context of the present invention are identified by polypeptide sequences (also called amino acid sequences herein). The polypeptide sequence specifies the three-dimensional structure including the “active site” of an enzyme which in turn determines the catalytic activity of the same. Polypeptide sequences may be identified by a SEQ ID NO. According to the World Intellectual Property Office (WIPO) Standard ST.25 (1998) the amino acids herein are represented using three-letter code with the first letter as a capital or the corresponding one letter.

The enzyme (component (b)) according to the invention relates to parent enzymes and/or variant enzymes, both having enzymatic activity. Enzymes having enzymatic activity are enzymatically active or exert enzymatic conversion, meaning that enzymes act on substrates and convert these into products. The term “enzyme” herein excludes inactive variants of an enzyme.

A “parent” sequence (of a parent protein or enzyme, also called “parent enzyme”) is the starting sequence for introduction of changes (e.g. by introducing one or more amino acid substitutions, insertions, deletions, or a combination thereof) to the sequence, resulting in “variants” of the parent sequences. The term parent enzyme (or parent sequence) includes wild-type enzymes (sequences) and synthetically generated sequences (enzymes) which are used as starting sequences for introduction of (further) changes.

The term “enzyme variant” or “sequence variant” or “variant enzyme” refers to an enzyme that differs from its parent enzyme in its amino acid sequence to a certain extent. If not indicated otherwise, variant enzyme “having enzymatic activity” means that this variant enzyme has the same type of enzymatic activity as the respective parent enzyme.

In describing the variants of the present invention, the nomenclature described as follows is used:

Amino acid substitutions are described by providing the original amino acid of the parent enzyme followed by the number of the position within the amino acid sequence, followed by the substituted amino acid.

Amino acid deletions are described by providing the original amino acid of the parent enzyme followed by the number of the position within the amino acid sequence, followed by *.

Amino acid insertions are described by providing the original amino acid of the parent enzyme followed by the number of the position within the amino acid sequence, followed by the original amino acid and the additional amino acid. For example, an insertion at position 180 of lysine next to glycine is designated as “Gly180GlyLys” or “G180GK”.

In cases where a substitution and an insertion occur at the same position, this may be indicated as S99SD+S99A or in short S99AD. In cases where an amino acid residue identical to the existing amino acid residue is inserted, it is clear that degeneracy in the nomenclature arises. If for example a glycine is inserted after the glycine in the above example this would be indicated by G180GG.

Where different alterations can be introduced at a position, the different alterations are separated by a comma, e.g. “Arg170Tyr, Glu” represents a substitution of arginine at position 170 with tyrosine or glutamic acid. Alternatively different alterations or optional substitutions may be indicated in brackets e.g. Arg170[Tyr, Gly] or Arg170{Tyr, Gly}; or in short R170 [Y,G] or R170 {Y, G}; or in long R170Y, R170G.

Enzyme variants may be defined by their sequence identity when compared to a parent enzyme. Sequence identity usually is provided as “% sequence identity” or “% identity”. For calculation of sequence identities, in a first step a sequence alignment has to be produced. According to this invention, a pairwise global alignment has to be produced, meaning that two sequences have to be aligned over their complete length, which is usually produced by using a mathematical approach, called alignment algorithm.

According to the invention, the alignment is generated by using the algorithm of Needleman and Wunsch (J. Mol. Biol. (1979) 48, p. 443-453). Preferably, the program “NEEDLE” (The European Molecular Biology Open Software Suite (EMBOSS)) is used for the purposes of the current invention, with using the programs default parameter (gap open=10.0, gap extend=0.5 and matrix=EBLOSUM62).

According to this invention, the following calculation of %-identity applies: %-identity=(identical residues/length of the alignment region which is showing the respective sequence of this invention over its complete length)*100.

According to this invention, enzyme variants may be described as an amino acid sequence which is at least n % identical to the amino acid sequence of the respective parent enzyme with “n” being an integer between 10 and 100. In one embodiment, variant enzymes are at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% identical when compared to the full length amino acid sequence of the parent enzyme, wherein the enzyme variant has enzymatic activity.

Enzyme variants may be defined by their sequence similarity when compared to a parent enzyme. Sequence similarity usually is provided as “% sequence similarity” or “%-similarity”. % sequence similarity takes into account that defined sets of amino acids share similar properties, e.g by their size, by their hydrophobicity, by their charge, or by other characteristics. Herein, the exchange of one amino acid with a similar amino acid may be called “conservative mutation”. For determination of %-similarity according to this invention the following applies: amino acid A is similar to amino acids S; amino acid D is similar to amino acids E and N; amino acid E is similar to amino acids D and K and Q; amino acid F is similar to amino acids W and Y; amino acid H is similar to amino acids N and Y; amino acid I is similar to amino acids L and M and V; amino acid K is similar to amino acids E and Q and R; amino acid L is similar to amino acids I and M and V; amino acid M is similar to amino acids I and L and V; amino acid N is similar to amino acids D and H and S; amino acid Q is similar to amino acids E and K and R; amino acid R is similar to amino acids K and Q; amino acid S is similar to amino acids A and N and T; amino acid T is similar to amino acids S; amino acid V is similar to amino acids I and L and M; amino acid W is similar to amino acids F and Y; amino acid Y is similar to amino acids F and H and W. Conservative amino acid substitutions may occur over the full length of the sequence of a polypeptide sequence of a functional protein such as an enzyme. In one embodiment, such mutations are not pertaining the functional domains of an enzyme. In one embodiment, conservative mutations are not pertaining the catalytic centers of an enzyme.

To take conservative mutations into account, a value for sequence similarity of two amino acid sequences may be calculated from the same alignment, which is used to calculate %-identity. According to this invention, the following calculation of %-similarity applies: %-similarity=[(identical residues+similar residues)/length of the alignment region which is showing the respective sequence(s) of this invention over its complete length]*100.

According to this invention, enzyme variants may be described as an amino acid sequence which is at least m % similar to the respective parent sequences with “m” being an integer between 10 and 100. In one embodiment, variant enzymes are at least 70%, at least 75%, at least 80%, at least 85%, at least 90%, at least 91%, at least 92%, at least 93%, at least 94%, at least 95%, at least 96%, at least 97%, at least 98%, or at least 99% similar when compared to the full length polypeptide sequence of the parent enzyme, wherein the variant enzyme has enzymatic activity.

“Enzymatic activity” means the catalytic effect exerted by an enzyme, which usually is expressed as units per milligram of enzyme (specific activity) which relates to molecules of substrate transformed per minute per molecule of enzyme (molecular activity).

Variant enzymes may have enzymatic activity according to the present invention when said enzyme variants exhibit at least 20%, at least 25%, at least 30%, at least 35%, at least 40%, at least 45%, at 10 least 50%, at least 55%, at least 60%, at least 65%, at least 70%, at least 75%, at least 80%, at least 85%, at least 90%, at least 95%, or 100% of the enzymatic activity of the respective parent enzyme.

### Protease

In one aspect of the invention, at least one enzyme comprised in component (b) is selected from the group of hydrolases (EC 3), preferably at least one enzyme selected from the group of proteases, more preferably at least one enzyme selected from the group of serine endopeptidases (EC 3.4.21), most preferably at least one enzyme selected from the group of subtilisin type proteases (EC 3.4.21.62).

Proteases are members of class EC 3.4. Proteases (component (b)) include aminopeptidases (EC 3.4.11), dipeptidases (EC 3.4.13), dipeptidyl-peptidases and tripeptidyl-peptidases (EC 3.4.14), peptidyl-dipeptidases (EC 3.4.15), serine-type carboxypeptidases (EC 3.4.16), metallo-carboxypeptidases (EC 3.4.17), cysteine-type carboxypeptidases (EC 3.4.18), omega peptidases (EC 3.4.19), serine endopeptidases (EC 3.4.21), cysteine endopeptidases (EC 3.4.22), aspartic endopeptidases (EC 3.4.23), metallo-endopeptidases (EC 3.4.24), threonine endopeptidases (EC 3.4.25), or endopeptidases of unknown catalytic mechanism (EC 3.4.99). In one embodiment, at least one protease (component (b)) is selected from serine proteases (EC 3.4.21). Serine proteases or serine peptidases are characterized by having a serine in the catalytically active site, which forms a covalent adduct with the substrate during the catalytic reaction. A serine protease (component (b)) in the context of the present invention is selected from the group consisting of chymotrypsin (e.g., EC 3.4.21.1), elastase (e.g., EC 3.4.21.36), elastase (e.g., EC 3.4.21.37 or EC 3.4.21.71), granzyme (e.g., EC 3.4.21.78 or EC 3.4.21.79), kallikrein (e.g., EC 3.4.21.34, EC 3.4.21.35, EC 3.4.21.118, or EC 3.4.21.119,) plasmin (e.g., EC 3.4.21.7), trypsin (e.g., EC 3.4.21.4), thrombin (e.g., EC 3.4.21.5), and subtilisin. Subtilisin is also known as subtilopeptidase, e.g., EC 3.4.21.62, the latter hereinafter also being referred to as “subtilisin”.

A sub-group of the serine proteases tentatively designated as subtilases has been proposed by Siezen et al. (1991), Protein Eng. 4:719-737 and Siezen et al. (1997), Protein Science 6:501-523. Subtilases includes the subtilisin family, thermitase family, the proteinase K family, the lantibiotic peptidase family, the kexin family and the pyrolysin family.

A subgroup of the subtilases are the subtilisins which are serine proteases from the family S8 as defined by the MEROPS database (http://merops.sanger.ac.uk). Peptidase family S8 comprises the serine endopeptidase subtilisin and its homologues. In subfamily S8A, the active site residues frequently occur in the motifs Asp-Thr/Ser-Gly (which is similar to the sequence motif in families of aspartic endopeptidases in clan AA), His-Gly-Thr-His and Gly-Thr-Ser-Met-Ala-Xaa-Pro.

The subtilisin related class of serine proteases (component (b)) shares a common amino acid sequence defining a catalytic triad which distinguishes them from the chymotrypsin related class of serine proteases. Subtilisins and chymotrypsin related serine proteases both have a catalytic triad comprising aspartate, histidine and serine.

Examples include the subtilisins as described in WO 89/06276 and EP 0283075, WO 89/06279, WO 89/09830, WO 89/09819, WO 91/06637 and WO 91/02792.

Proteases are active proteins exerting “protease activity” or “proteolytic activity”. Proteolytic activity is related to the rate of degradation of protein by a protease or proteolytic enzyme in a defined course of time.

The methods for analyzing proteolytic activity are well-known in the literature (see e.g. Gupta et al. (2002), Appl. Microbiol. Biotechnol. 60: 381-395). Proteolytic activity may be determined by using Succinyl-Ala-Ala-Pro-Phe-p-nitroanilide (Suc-AAPF-pNA, short AAPF; see e.g. DelMar et al. (1979), Analytical Biochem 99, 316-320) as substrate. pNA is cleaved from the substrate molecule by proteolytic cleavage, resulting in release of yellow color of free pNA which can be quantified by measuring OD₄₀₅.

Proteolytic activity may be provided in units per gram enzyme. For example, 1 U protease may correspond to the amount of protease which sets free 1 μmol folin-positive amino acids and peptides (as tyrosine) per minute at pH 8.0 and 37° C. (casein as substrate).

Proteases (component (b)) of the subtilisin type (EC 3.4.21.62) may be bacterial proteases originating from a microorganism selected from Bacillus, Clostridium, Enterococcus, Geobacillus, Lactobacillus, Lactococcus, Oceanobacillus, Staphylococcus, Streptococcus, or Streptomyces protease, or a Gram-negative bacterial polypeptide such as a Campylobacter, E. coli, Flavobacterium, Fusobacterium, Helicobacter, Ilyobacter, Neisseria, Pseudomonas, Salmonella, and Ureaplasma.

In one aspect of the invention, at least one protease (component (b)) is selected from Bacillus alcalophllus, Bacillus amyloliquefaciens, Bacillus brevis, Bacillus circulars, Bacillus clausii, Bacillus coagulans, Bacillus firmus, Bacillus gibsonii, Bacillus lautus, Bacillus lentus, Bacillus licheniformis, Bacillus megaterium, Bacillus pumllus, Bacillus sphaericus, Bacillus stearothermophilus, Bacillus subtilis, or Bacillus thuringiensis protease.

In one embodiment of the present invention, at least one protease (component (b)) is selected from the following: subtilisin from Bacillus amyloliquefaciens BPN′ (described by Vasantha et al. (1984) J. Bacteriol. Volume 159, p. 811-819 and JA Wells et al. (1983) in Nucleic Acids Research, Volume 11, p. 7911-7925); subtilisin from Bacillus licheniformis (subtilisin Carlsberg; disclosed in E L Smith et al. (1968) in J. Biol Chem, Volume 243, pp. 2184-2191, and Jacobs et al. (1985) in Nucl. Acids Res, Vol 13, p. 8913-8926); subtilisin PB92 (original sequence of the alkaline protease PB92 is described in EP 283075 A2); subtilisin 147 and/or 309 (Esperase®, Savinase®, respectively) as disclosed in WO 89/06279; subtilisin from Bacillus lentus as disclosed in WO 91/02792, such as from Bacillus lentus DSM 5483 or the variants of Bacillus lentus DSM 5483 as described in WO 95/23221; subtilisin from Bacillus alcalophilus (DSM 11233) disclosed in DE 10064983; subtilisin from Bacillus gibsonii (DSM 14391) as disclosed in WO 2003/054184; subtilisin from Bacillus sp. (DSM 14390) disclosed in WO 2003/056017; subtilisin from Bacillus sp. (DSM 14392) disclosed in WO 2003/055974; subtilisin from Bacillus gibsonii (DSM 14393) disclosed in WO 2003/054184; subtilisin having SEQ ID NO: 4 as described in WO 2005/063974; subtilisin having SEQ ID NO: 4 as described in WO 2005/103244; subtilisin having SEQ ID NO: 7 as described in WO 2005/103244; and subtilisin having SEQ ID NO: 2 as described in application DE 102005028295.4.

In one embodiment, component (b) comprises at least subtilisin 309 (which might be called Savinase herein) as disclosed as sequence a) in Table I of WO 89/06279 or a variant thereof which is at least 80% identical thereto and has proteolytic activity.

Examples of useful proteases (component (b)) in accordance with the present invention comprise the variants described in: WO 92/19729, WO 95/23221, WO 96/34946, WO 98/20115, WO 98/20116, WO 99/11768, WO 01/44452, WO 02/088340, WO 03/006602, WO 2004/03186, WO 2004/041979, WO 2007/006305, WO 2011/036263, WO 2011/036264, and WO 2011/072099. Suitable examples comprise especially variants of subtilisin protease derived from SEQ ID NO:22 as described in EP 1921147 (which is the sequence of mature alkaline protease from Bacillus lentus DSM 5483) with amino acid substitutions in one or more of the following positions: 3, 4, 9, 15, 24, 27, 33, 36, 57, 68, 76, 77, 87, 95, 96, 97, 98, 99, 100, 101, 102, 103, 104, 106, 118, 120, 123, 128, 129, 130, 131, 154, 160, 167, 170, 194, 195, 199, 205, 206, 217, 218, 222, 224, 232, 235, 236, 245, 248, 252 and 274 (according to the BPN′ numbering), which have proteolytic activity. In one embodiment, such a protease is not mutated at positions Asp32, His64 and Ser221 (according to BPN′ numbering).

Suitable proteases (component (b)) include protease variants having proteolytic activity which are at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% identical when compared to the full length polypeptide sequence of the parent enzyme as disclosed above.

Suitable proteases (component (b)) include protease variants having proteolytic activity which are at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% similar when compared to the full length polypeptide sequence of the parent enzyme.

In one embodiment, at least one protease (component (b)) has SEQ ID NO:22 as described in EP 1921147, or a protease which is at least 80% identical thereto and has proteolytic activity. In one embodiment, said protease is characterized by having amino acid glutamic acid (E), or aspartic acid (D), or asparagine (N), or glutamine (Q), or alanine (A), or glycine (G), or serine (S) at position 101 (according to BPN′ numbering) and has proteolytic activity. In one embodiment, said protease comprises one or more further substitutions: (a) threonine at position 3 (3T), (b) isoleucine at position 4 (4I), (c) alanine, threonine or arginine at position 63 (63A, 63T, or 63R), (d) aspartic acid or glutamic acid at position 156 (156D or 156E), (e) proline at position 194 (194P), (f) methionine at position 199 (199M), (g) isoleucine at position 205 (205I), (h) aspartic acid, glutamic acid or glycine at position 217 (217D, 217E or 217G), (i) combinations of two or more amino acids according to (a) to (h). At least one protease (component (b)) may be at least 80% identical to SEQ ID NO:22 as described in EP 1921147 and is characterized by comprising one amino acid (according to (a)-(h)) or combinations according to (i) together with the amino acid 101E, 101D, 101N, 101Q, 101A, 101G, or 101S (according to BPN′ numbering) and having proteolytic activity. In one embodiment, said protease is characterized by comprising the mutation (according to BPN′ numbering) R101E, or S3T+V4I+V205I, or R101E and S3T, V4I, and V205I, or S3T+V4I+V199M+V205I+L217D, and having proteolytic activity.

In one embodiment, protease according to SEQ ID NO:22 as described in EP 1921147 is characterized by comprising the mutation (according to BPN′ numbering) S3T+V4I+S9R+A15T+V68A+D99S+R101S+A103S+I104V+N218D, and having proteolytic activity.

In one embodiment, at least one protease is selected from commercially available protease enzymes which include but are not limited to products sold under the trade names Alcalase®, Blaze®, Duralase™, Durazym™, Relase®, Relase® Ultra, Savinase®, Savinase® Ultra, Primase®, Polarzyme®, Kannase®, Liquanase®, Liquanase® Ultra, Ovozyme®, Coronase®, Coronase® Ultra, Neutrase®, Everlase® and Esperase® (Novozymes NS), those sold under the tradename Maxatase®, Maxacal®, Maxapem®, Purafect®, Purafect® Prime, Purafect MA®, Purafect Ox®, Purafect OxP®, Puramax®, Properase®, FN2®, FN3®, FN4®, Excellase®, Eraser®, Ultimase®, Opticlean®, Effectenz®, Preferenz® and Optimase® (Danisco/DuPont), Axapem™ (Gist-Brocases N.V.), Bacillus lentus Alkaline Protease (BLAP; sequence shown in FIG. 29 of U.S. Pat. No. 5,352,604) and variants thereof and KAP (Bacillus alkalophilus subtilisin) from Kao Corp.

According to the present invention, component (b) may comprise a combination of at least two proteases, preferably selected from the group of serine endopeptidases (EC 3.4.21), more preferably selected from the group of subtilisin type proteases (EC 3.4.21.62)—all as disclosed above.

In one embodiment, component (b) comprises at least one protease selected from proteases according to SEQ ID NO:22 as described in EP 1921147 or variants thereof having proteolytic activity, as disclosed above.

In one embodiment, component (b) comprises at least one protease selected from subtilisin 309 as disclosed in Table I a) of WO 89/06279 or variants thereof having proteolytic activity, as disclosed above.

In one embodiment, component (b) comprises a combination of at least one protease, preferably selected from the group of serine endopeptidases (EC 3.4.21), more preferably selected from the group of subtilisin type proteases (EC 3.4.21.62), and at least one lipase.

### Lipase

“Lipases”, “lipolytic enzyme”, “lipid esterase”, all refer to an enzyme of EC class 3.1.1 (“carboxylic ester hydrolase”). Lipase means active protein having lipase activity (or lipolytic activity; triacylglycerol lipase, EC 3.1.1.3), cutinase activity (EC 3.1.1.74; enzymes having cutinase activity may be called cutinase herein), sterol esterase activity (EC 3.1.1.13) and/or wax-ester hydrolase activity (EC 3.1.1.50).

The methods for determining lipolytic activity are well-known in the literature (see e.g. Gupta et al. (2003), Biotechnol. Appl. Biochem. 37, p. 63-71). E.g. the lipase activity may be measured by ester bond hydrolysis in the substrate para-nitrophenyl palmitate (pNP-Palmitate, C:16) and releases pNP which is yellow and can be detected at 405 nm.

“Lipolytic activity” means the catalytic effect exerted by a lipase, which may be provided in lipolytic units (LU). For example, 1 LU may correspond to the amount of lipase which produces 1 μmol of titratable fatty acid per minute in a pH stat. under the following conditions: temperature 30° C.; pH=9.0; substrate may be an emulsion of 3.3 wt. % of olive oil and 3.3% gum arabic, in the presence of 13 mmol/l Ca²⁺ and 20 mmol/1 NaCl in 5 mmol/l Tris-buffer.

Lipases (component (b)) include those of bacterial or fungal origin. In one aspect of the invention, a suitable lipase (component (b)) is selected from the following: lipases from Humicola (synonym Thermomyces), e.g. from H. lanuginosa (T. lanuginosus) as described in EP 258068, EP 305216, WO 92/05249 and WO 2009/109500 or from H. insolens as described in WO 96/13580; lipases derived from Rhizomucor miehei as described in WO 92/05249; lipase from strains of Pseudomonas (some of these now renamed to Burkholderia), e.g. from P. alcaligenes or P. pseudoalcaligenes (EP 218272, WO 94/25578, WO 95/30744, WO 95/35381, WO 96/00292), P. cepacia (EP 331376), P. stutzeri (GB 1372034), P. fluorescens, Pseudomonas sp. strain S D705 (WO 95/06720 and WO 96/27002), P. wisconsinensis (WO 96/12012), Pseudomonas mendocina (WO 95/14783), P. glumae (WO 95/35381, WO 96/00292); lipase from Streptomyces griseus (WO 2011/150157) and S. pristinaespiralis (WO 2012/137147), GDSL-type Streptomyces lipases (WO 2010/065455); lipase from Thermobifida fusca as disclosed in WO 2011/084412; lipase from Geobacillus stearothermophilus as disclosed in WO 2011/084417; Bacillus lipases, e.g. as disclosed in WO 00/60063, lipases from B. subtilis as disclosed in Dartois et al. (1992), Biochemica et Biophysica Acta, 1131, 253-360 or WO 2011/084599, B. stearothermophilus (JP S64-074992) or B. pumilus (WO 91/16422); lipase from Candida antarctica as disclosed in WO 94/01541; cutinase from Pseudomonas mendocina (U.S. Pat. No. 5,389,536, WO 88/09367); cutinase from Magnaporthe grisea (WO 2010/107560); cutinase from Fusarium solani pisi as disclosed in WO 90/09446, WO 00/34450 and WO 01/92502; and cutinase from Humicola lanuginosa as disclosed in WO 00/34450 and WO 01/92502.

Suitable lipases (component (b)) also include those referred to as acyltransferases or perhydrolases, e.g. acyltransferases with homology to Candida antarctica lipase A (WO 2010/111143), acyltransferase from Mycobacterium smegmatis (WO 2005/056782), perhydrolases from the CE7 family (WO 2009/67279), and variants of the M. smegmatis perhydrolase in particular the S54V variant (WO 2010/100028).

Suitable lipases (component (b)) include also those which are variants of the above described lipases which have lipolytic activity. Such suitable lipase variants (component (b)) are e.g. those which are developed by methods as disclosed in WO 95/22615, WO 97/04079, WO 97/07202, WO 00/60063, WO 2007/087508, EP 407225 and EP 260105.

Suitable lipases (component (b)) include lipase variants having lipolytic activity which are at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% identical when compared to the full length polypeptide sequence of the parent enzyme as disclosed above.

Suitable lipases (component (b)) include lipase variants having lipolytic activity which are at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% similar when compared to the full length polypeptide sequence of the parent enzyme.

In one embodiment, at least one lipase (component (b)) is selected from fungal triacylglycerol lipase (EC class 3.1.1.3). Fungal triacylglycerol lipase (component (b)) may be selected from Thermomyces lanuginose lipase. In one embodiment, Thermomyces lanuginosa lipase (component (b)) is selected from triacylglycerol lipase according to amino acids 1-269 of SEQ ID NO:2 of U.S. Pat. No. 5,869,438 and variants thereof having lipolytic activity. Triacylglycerol lipase according to amino acids 1-269 of SEQ ID NO:2 of U.S. Pat. No. 5,869,438 may be called Lipolase herein.

Thermomyces lanuginosa lipase (component (b)) may be selected from variants having lipolytic activity which are at least 90%, at least 91%, at least 92%, at least 93%, at least 94%, at least 95%, at least 96%, at least 97%, at least 98% or at least 99% identical when compared to the full length polypeptide sequence of amino acids 1-269 of SEQ ID NO:2 of U.S. Pat. No. 5,869,438.

Thermomyces lanuginosa lipase (component (b)) may be selected from variants having lipolytic activity comprising conservative mutations only, which do however not pertain the functional domain of amino acids 1-269 of SEQ ID NO:2 of U.S. Pat. No. 5,869,438. Lipase variants of this embodiment having lipolytic activity may be at least 95%, at least 96%, at least 97%, at least 98% or at least 99% similar when compared to the full length polypeptide sequence of amino acids 1-269 of SEQ ID NO:2 of U.S. Pat. No. 5,869,438.

Thermomyces lanuginosa lipase (component (b)) may be at least 80% identical to SEQ ID NO:2 of U.S. Pat. No. 5,869,438 characterized by having amino acid T231R and N233R. Said Thermomyces lanuginosa lipase may further comprise one or more of the following amino acid exchanges: Q4V, V605, A150G, L227G, P256K.

In one embodiment, at least one lipase is selected from commercially available lipases which include but are not limited to products sold under the trade names Lipolase™, Lipex™, Lipolex™and Lipoclean™ (Novozymes NS), Lumafast (originally from Genencor) and Lipomax (Gist-Brocades/ now DSM).

According to the present invention, component (b) may comprise a combination of at least two lipases, preferably selected from triacylglycerol lipase according to amino acids 1-269 of SEQ ID NO:2 of U.S. Pat. No. 5,869,438 and variants thereof having lipolytic activity as disclosed above.

In one embodiment, component (b) comprises at least one protease selected from the group of serine endopeptidases (EC 3.4.21), preferably selected from the group of subtilisin type proteases (EC 3.4.21.62), and at least one triacylglycerol lipase according to amino acids 1-269 of SEQ ID NO:2 of U.S. Pat. No. 5,869,438 or a variant thereof having lipolytic activity as disclosed above.

In one embodiment, component (b) comprises at least one protease selected from proteases according to SEQ ID NO:22 as described in EP 1921147 or variants thereof having proteolytic activity, and at least one triacylglycerol lipase according to amino acids 1-269 of SEQ ID NO:2 of U.S. Pat. No. 5,869,438 or a variant thereof having lipolytic activity—all as disclosed above.

In one embodiment, component (b) comprises at least one protease selected from subtilisin 309 as disclosed in Table I a) of WO 89/06279 or variants thereof having proteolytic activity, and at least one triacylglycerol lipase according to amino acids 1-269 of SEQ ID NO:2 of U.S. Pat. No. 5,869,438 or a variant thereof having lipolytic activity—all as disclosed above.

In one embodiment, component (b) comprises a combination of at least one protease, preferably selected from the group of serine endopeptidases (EC 3.4.21), more preferably selected from the group of subtilisin type proteases (EC 3.4.21.62), and at least one amylase.

### Amylase

In one embodiment, inventive enzyme preparations comprise at least one amylase (component (b)). “Amylases” (component (b)) according to the invention (alpha and/or beta) include those of bacterial or fungal origin (EC 3.2.1.1 and 3.2.1.2, respectively). Chemically modified or protein engineered mutants are included.

Amylases (component (b)) according to the invention have “amylolytic activity” or “amylase activity” involving (endo)hydrolysis of glucosidic linkages in polysaccharides. α-amylase activity may be determined by assays for measurement of α-amylase activity which are known to those skilled in the art. Examples for assays measuring α-amylase activity are:

α-amylase activity can be determined by a method employing Phadebas tablets as substrate (Phadebas Amylase Test, supplied by Magle Life Science). Starch is hydrolyzed by the α-amylase giving soluble blue fragments. The absorbance of the resulting blue solution, measured spectrophotometrically at 620 nm, is a function of the α-amylase activity. The measured absorbance is directly proportional to the specific activity (activity/mg of pure α-amylase protein) of the α-amylase in question under the given set of conditions.

α-amylase activity can also be determined by a method employing the Ethyliden-4-nitrophenyl-α-D-maltoheptaosid (EPS). D-maltoheptaoside is a blocked oligosaccharide which can be cleaved by an endo-amylase. Following the cleavage, the α-glucosidase included in the kit to digest the substrate to liberate a free PNP molecule which has a yellow color and thus can be measured by visible spectophotometry at 405 nm. Kits containing EPS substrate and α-glucosidase is manufactured by Roche Costum Biotech (cat. No. 10880078103). The slope of the time dependent absorption-curve is directly proportional to the specific activity (activity per mg enzyme) of the α-amylase in question under the given set of conditions.

Amylolytic activity may be provided in units per gram enzyme. For example, 1 unit α-amylase may liberate 1.0 mg of maltose from starch in 3 min at pH 6.9 at 20° C.

At least one amylase (component (b)) may be selected from the following: amylases from Bacillus licheniformis having SEQ ID NO:2 as described in WO 95/10603; amylases from B. stearothermophilus having SEQ ID NO:6 as disclosed in WO 02/10355; amylases from Bacillus sp. 707 having SEQ ID NO:6 as disclosed in WO 99/19467; amylases from Bacillus halmapalus having SEQ ID NO:2 or SEQ ID NO:7 as described in WO 96/23872, also described as SP-722; amylases from Bacillus sp. DSM 12649 having SEQ ID NO:4 as disclosed in WO 00/22103; amylases from Bacillus strain TS-23 having SEQ ID NO:2 as disclosed in WO 2009/061380; amylases from Cytophaga sp. having SEQ ID NO:1 as disclosed in WO 2013/184577; amylases from Bacillus megaterium DSM 90 having SEQ ID NO:1 as disclosed in WO 2010/104675; amylases from Bacillus sp. comprising amino acids 1 to 485 of SEQ ID NO:2 as described in WO 00/60060.

Suitable amylases (component (b)) include amylase variants having amylase activity which are at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% identical when compared to the full length polypeptide sequence of the parent enzyme as disclosed above.

Suitable amylases (component (b)) include amylase variants having amylase activity which are at least 40%, at least 45%, at least 50%, at least 55%, at least 60%, at least 65%, at least 70%, at least 75%, at least 80%, at least 85%, at least 90%, at least 91%, at least 92%, at least 93%, at least 94%, at least 95%, at least 96%, at least 97%, at least 98% or at least 99% similar when compared to the full length polypeptide sequence of the parent enzyme.

At least one amylase (component (b)) may have SEQ ID NO: 12 as described in WO 2006/002643 or is at least 80% identical thereto and has amylolytic activity. At least one amylase may be at least 80% identical to SEQ ID NO:12 and comprises the substitutions at positions Y295F and M202LITV.

At least one amylase (component (b)) may have SEQ ID NO:6 as described in WO 2011/098531 or is at least 80% identical thereto and has amylolytic activity. At least one amylase may be at least 80% identical to SEQ ID NO:6 and comprises a substitution at one or more positions selected from the group consisting of 193 [G,A,S,T or M], 195 [F,W,Y,L,I or V], 197 [F,W,Y,L,I or V], 198 [Q or N], 200 [F,W,Y,L,I or V], 203 [F,W,Y,L,I or V], 206 [F,W,Y,N,L,I,V,H,Q,D or E], 210 [F,W,Y,L,I or V], 212 [F,W,Y,L,I or V], 213 [G,A,S,T or M] and 243 [F,W,Y,L,I or V].

At least one amylase (component (b)) may have SEQ ID NO:1 as described in WO 2013/001078 or is at least 85% identical thereto and has amylolytic activity. At least one amylase may be at least 85% identical to SEQ ID NO:1 and comprises an alteration at two or more (several) positions corresponding to positions G304, W140, W189, D134, E260, F262, W284, W347, W439, W469, G476, and G477.

At least one amylase (component (b)) may have SEQ ID NO:2 as described in WO 2013/001087 or is at least 85% identical thereto and has amylolytic activity. At least one amylase may be at least 85% identical to SEQ ID NO:2 and comprises a deletion of positions 181+182, or 182+183, or 183+184, and has amylolytic activity. In one embodiment, said amylase may comprise one or two or more further modifications in any of positions corresponding to W140, W159, W167, Q169, W189, E194, N260, F262, W284, F289, G304, G305, R320, W347, W439, W469, G476 and G477.

In one embodiment, at least one amylase is selected from commercially available amylases which include but are not limited to products sold under the trade names Duramyl™, Termamyl™, Fungamyl™, Stainzyme™, Stainzyme Plus™, Natalase™, Liquozyme X and BAN™ (from Novozymes NS), and Rapidase™, Purastar™, Powerase™, Effectenz™ (M100 from DuPont), Preferenz™ (S1000, S110 and F1000; from DuPont), PrimaGreen™ (ALL; DuPont), Optisize™ (DuPont).

According to the present invention, component (b) may comprise a combination of at least two amylases.

In one embodiment, component (b) comprises a combination of at least one protease and at least one amylase.

In one embodiment, component (b) comprises a combination of at least one protease and at least one lipase and at least one amylase.

### Cellulase

In one embodiment, component (b) comprises a combination of at least one protease, preferably selected from the group of serine endopeptidases (EC 3.4.21), more preferably selected from the group of subtilisin type proteases (EC 3.4.21.62), and at least one cellulase.

The enzyme preparation of the invention may comprise at least one cellulase (component (b)). Three major types of cellulases are known, namely cellobiohydrolase (1,4-P-D-glucan cellobiohydrolase, EC 3.2.1.91), endo-ss-1,4-glucanase (endo-1,4-P-D-glucan 4-glucanohydrolase, EC 3.2.1.4) and ss-glucosidase (EC 3.2.1.21).

“Cellulases”, “cellulase enzymes” or “cellulolytic enzymes” (component (b)) are enzymes involved in hydrolysis of cellulose. Assays for measurement of “cellulase activity” or “cellulolytic activity” are known to those skilled in the art. For example, cellulolytic activity may be determined by virtue of the fact that cellulase hydrolyses carboxymethyl cellulose to reducing carbohydrates, the reducing ability of which is determined colorimetrically by means of the ferricyanide reaction, according to Hoffman, W. S., J. Biol. Chem. 120, 51 (1937).

Cellulolytic activity may be provided in units per gram enzyme. For example, 1 unit may liberate 1.0 μmole of glucose from cellulose in one hour at pH 5.0 at 37° C. (2 hour incubation time).

Cellulases according to the invention include those of bacterial or fungal origin. In one embodiment, at least one cellulase is selected from cellulases comprising a cellulose binding domain. In one embodiment, at least one cellulase is selected from cellulases comprising a catalytic domain only, meaning that the cellulase lacks cellulose binding domain.

In one embodiment, at least one cellulase (component (b)) is selected from commercially available cellulases which include but are not limited to Celluzyme™, Endolase™, Carezyme™, Cellusoft™, Renozyme™, Celluclean™ (from Novozymes NS), Ecostone™, Biotouch™, Econase™, Ecopulp™ (from AB Enzymes Finland), Clazinase™, and Puradax HA™, Genencor detergent cellulase L, IndiAge™ Neutra (from Genencor International Inc./DuPont), Revitalenz™ (2000 from DuPont), Primafast™ (DuPont) and KAC500™ (from Kao Corporation).

According to the present invention, component (b) may comprise a combination of at least two cellulases.

In one embodiment, component (b) comprises a combination of at least one protease and at least one cellulase.

In one embodiment, component (b) comprises a combination of at least one protease and at least one lipase and at least one cellulase.

In one embodiment, component (b) comprises a combination of at least one protease and at least one amylase and at least one cellulase.

In one embodiment, component (b) comprises a combination of at least one protease and at least one lipase and at least one amylase and at least one cellulase.

### Component (c)

In one embodiment, the liquid enzyme preparation of the invention comprises component (c) which comprises at least one compound selected from solvents, enzyme stabilizers different from component (a), and compounds stabilizing the liquid enzyme preparation as such.

Enzyme Stabilizers Different from Component (a):

The liquid enzyme preparation of the invention may comprise at least one enzyme stabilizer different from component (a). Said enzyme stabilizer (component (c)) may be selected from boron-containing compounds, polyols, peptide aldehydes, other stabilizers, and mixtures thereof.

### Boron-Containing Compounds:

Boron-containing compounds (component (c)) may be selected from boric acid or its derivatives and from boronic acid or its derivatives such as aryl boronic acids or its derivatives, from salts thereof, and from mixtures thereof. Boric acid herein may be called orthoboric acid.

In one embodiment, boron-containing compound (component (c)) is selected from the group consisting of aryl boronic acids and its derivatives. In one embodiment, boron-containing compound is selected from the group consisting of benzene boronic acid (BBA) which is also called phenyl boronic acid (PBA), derivatives thereof, and mixtures thereof. In one embodiment, phenyl boronic acid derivatives are selected from the group consisting of the derivatives of formula (IIIa) and (IIIb) formula:

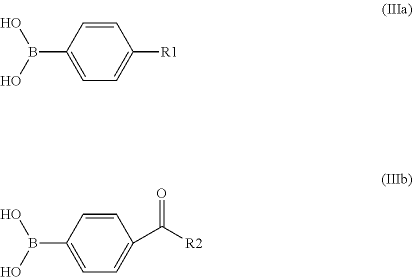

wherein

R1 is selected from the group consisting of hydrogen, hydroxy, non-substituted or substituted C₁-C₆ alkyl, and non-substituted or substituted C₁-C₆ alkenyl; in a preferred embodiment, R is selected from the group consisting of hydroxy, and non-substituted C₁ alkyl;

R2 is selected from the group consisting of hydrogen, hydroxy, non-substituted or substituted C₁-C₆ alkyl, and non-substituted or substituted C₁-C₆ alkenyl; in a preferred embodiment, R is selected from the group consisting of H, hydroxy, and substituted C₁ alkyl.

In one embodiment phenyl-boronic acid derivatives (component (c)) are selected from the group consisting of 4-formyl phenyl boronic acid (4-FPBA), 4-carboxy phenyl boronic acid (4-CPBA), 4-(hydroxymethyl) phenyl boronic acid (4-HMPBA), and p-tolylboronic acid (p-TBA).

Other suitable derivatives (component (c)) include: 2-thienyl boronic acid, 3-thienyl boronic acid, (2-acetamidophenyl) boronic acid, 2-benzofuranyl boronic acid, 1-naphthyl boronic acid, 2-naphthyl boronic acid, 2-FPBA, 3-FBPA, 1-thianthrenyl boronic acid, 4-dibenzofuran boronic acid, 5-methyl-2-thienyl boronic acid, 1-benzothiophene-2 boronic acid, 2-furanyl boronic acid, 3-furanyl boronic acid, 4,4 biphenyl-diboronic acid, 6-hydroxy-2-naphthaleneboronic acid, 4-(methylthio) phenyl boronic acid, 4-(trimethylsilyl) phenyl boronic acid, 3-bromothiophene boronic acid, 4-methylthiophene boronic acid, 2-naphthyl boronic acid, 5-bromothiophene boronic acid, 5-chlorothiophene boronic acid, dimethylthiophene boronic acid, 2-bromophenyl boronic acid, 3-chlorophenyl boronic acid, 3-methoxy-2-thiophene boronic acid, p-methyl-phenylethyl boronic acid, 2-thianthrenyl boronic acid, di-benzothiophene boronic acid, 9-anthracene boronic acid, 3,5 dichlorophenyl boronic, acid, diphenyl boronic acid anhydride, o-chlorophenyl boronic acid, p-chlorophenyl boronic acid, m-bromophenyl boronic acid, p-bromophenyl boronic acid, p-fluorophenyl boronic acid, octyl boronic acid, 1,3,5 trimethylphenyl boronic acid, 3-chloro-4-fluorophenyl boronic acid, 3-aminophenyl boronic acid, 3,5-bis-(trifluoromethyl) phenyl boronic acid, 2,4 dichlorophenyl boronic acid, 4-methoxyphenyl boronic acid, and mixtures thereof.

### Polyols:

Polyols (component (c)) may be selected from polyols containing from 2 to 6 hydroxyl groups. Suitable examples include glycol, propylene glycol, 1,2-propane diol, 1,2-butane diol, ethylene glycol, hexylene glycol, glycerol, sorbitol, mannitol, erythritol, glucose, fructose, lactore, and erythrinan.

### Peptide Aldehydes:

Peptide aldehydes (component (c)) may be selected from di-, tri- or tetrapeptide aldehydes and aldehyde analogues (either of the form B1-BO—R wherein, R is H, CHs, CX₃, CHX₂, or CH₂X (X=halogen), BO is a single amino acid residue (in one embodiment with an optionally substituted aliphatic or aromatic side chain); and B1 consists of one or more amino acid residues (in one embodiment one, two or three), optionally comprising an N-terminal protection group, or as described in WO 09/118375 and WO 98/13459, or a protease inhibitor of the protein type such as RASI, BASI, WASI (bifunctional alpha-amylase/subtilisin inhibitors of rice, barley and wheat) or Cl₂ or SSI.

### Other Stabilizers:

Other stabilizers (component (c)) may be selected from salts like NaCl or KCl, and alkali salts of lactic acid and formic acid.

Other stabilizers (component (c)) may be selected from water-soluble sources of zinc (II), calcium (II) and/or magnesium (II) ions in the finished compositions that provide such ions to the enzymes, as well as other metal ions (e.g. barium (II), scandium (II), iron (II), manganese (II), aluminum (III), Tin (II), cobalt (II), copper (II), Nickel (II), and oxovanadium (IV)).

### Compounds Stabilizing the Liquid Enzyme Preparation as Such

Compounds stabilizing the liquid enzyme preparation as such means any compound except enzyme stabilizers needed to establish storage stability of a liquid preparation in amounts effective to ensure the storage stability.

Storage stability in the context of liquid preparations to those skilled in the art usually includes aspects of appearance of the product and uniformity of dosage.

Appearance of the product is influenced by the pH of the product and by the presence of compounds such as preservatives, antioxidants, viscosity modifiers, emulsifiers etc.

Uniformity of dosage is usually related to the homogeneity of a product.

Inventive enzyme preparations may be alkaline or exhibit a neutral or slightly acidic pH value, for example 6 to 14, 6.5 to 13, 8 to 10.5, or 8.5 to 9.0.

The liquid enzyme preparation of the invention may comprise at least one preservative. Preservatives are added in amounts effective in preventing microbial contamination of the liquid enzyme preparation, preferably the aqueous enzyme preparation.

Non-limiting examples of suitable preservatives include (quaternary) ammonium compounds, isothiazolinones, organic acids, and formaldehyde releasing agents. Non-limiting examples of suitable (quaternary) ammonium compounds include benzalkonium chlorides, polyhexamethylene biguanide (PHMB), Didecyldimethylammonium chloride (DDAC), and N-(3-aminopropyl)-N-dodecylpropane-1,3-diamine (Diamine). Non-limiting examples of suitable isothiazolinones include 1,2-benzisothiazolin-3-one (BIT), 2-methyl-2H-isothiazol-3-one (MIT), 5-chloro-2-methyl-2H-isothiazol-3-one (CIT), 2-octyl-2H-isothiazol-3-one (OIT), and 2-butyl-benzo[d]isothiazol-3-one (BBIT). Non-limiting examples of suitable organic acids include benzoic acid, sorbic acid, L(+)-lactic acid, formic acid, and salicylic acid. Non-limiting examples of suitable formaldehyde releasing agent include N,N′-methylenebismorpholine (MBM), 2,2′,2″-(hexahydro-1,3,5-triazine-1,3,5-triyl)triethanol (HHT), (ethylenedioxy)dimethanol, .alpha.,.alpha.′,.alpha.″-trimethyl-1,3,5-triazine-1,3,5(2H,4H,6H)-triethanol (HPT), 3,3′-methylenebis[5-methyloxazolidine] (MBO), and cis-1-(3-chloroallyl)-3,5,7-triaza-1-azoniaadamantane chloride (CTAC).

Further useful preservatives include iodopropynyl butylcarbamate (IPBC), halogen releasing compounds such as dichloro-dimethyl-hydantoine (DCDMH), bromo-chloro-dimethyl-hydantoine (BCDMH), and dibromo-dimethyl-hydantoine (DBDMH); bromo-nitro compounds such as Bronopol (2-bromo-2-nitropropane-1,3-diol), 2,2-dibromo-2-cyanoacetamide (DBNPA); aldehydes such as glutaraldehyde; phenoxyethanol; Biphenyl-2-ol; and zinc or sodium pyrithione.

### Solvents

In one embodiment, the inventive enzyme preparation is aqueous, comprising water in amounts in the range of 5% to 95% by weight, in the range of 5% to 30% by weight, in the range of 5% to 25% by weight, or in the range of 20% to 70% by weight, all relative to the total weight of the enzyme preparation.

In one embodiment, the enzyme preparation of the invention comprises at least one organic solvent selected from ethanol, n-propanol, iso-propanol, n-butanol, iso-butanol, sec.-butanol, ethylene glycol, propylene glycol, 1,3-propane diol, butane diol, glycerol, diglycol, propyl diglycol, butyl diglycol, hexylene glycol, ethylene glycol methyl ether, ethylene glycol ethyl ether, ethylene glycol propyl ether, and phenoxyethanol, preferred are ethanol, isopropanol or propylene glycol. Further, the enzyme preparation of the invention may comprise at least one organic solvent selected from compounds such as 2-butoxyethanol, isopropyl alcohol, and d-limonene. Said enzyme preparation may comprise organic solvents in amounts in the range of 0% to 20% by weight relative to the total weight of the enzyme preparation. In one embodiment, the enzyme preparation comprises water in amounts in the range of 5% to 15% by weight and no significant amounts of organic solvent, for example 1% by weight or less, all relative to the total weight of the enzyme preparation.

In one embodiment, the enzyme preparation of the invention comprises at least
component (a): at least one enzyme stabilizer selected from compounds according to general formula (I)

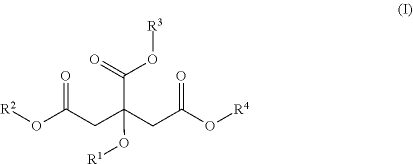

wherein the variables in formula (I) are as follows:
R¹ is selected from H and C₁-C₁₀ alkylcarbonyl, wherein alkyl may be linear or branched and may bear one or more hydroxyl groups,
R², R³, R⁴ are independently from each other selected from H, linear C₁-C₅ alkyl, and branched C₃-C₁₀ alkyl, C₆-C₁₀-aryl, non-substituted or substituted with one or more carboxylate or hydroxyl groups, and C₆-C₁₀-aryl-alkyl, wherein alkyl of the latter is selected from linear C₁-C₈ alkyl or branched C₃-C₈ alkyl, wherein at least one of R², R³, and R⁴ is not H, and
component (b): at least one enzyme selected from the group of proteases, more preferably at least one enzyme selected from the group of serine endopeptidases (EC 3.4.21), most preferably at least one enzyme selected from the group of subtilisin type proteases (EC 3.4.21.62); and optionally at least one enzyme selected from the group of lipases and amylases;
and
component (c): at least one enzyme stabilizer different from component (a), preferably selected from boron containing compounds as disclosed above, more preferably selected from phenyl boronic acid (PBA) or its derivatives as disclosed above, most preferably being 4-formyl phenyl boronic acid (4-FPBA).

### Preparation of Enzyme Preparation

The invention relates to a process for making an enzyme preparation, said process comprising the step of mixing at least component (a) as disclosed above and component (b) as disclosed above.

In one embodiment the invention relates to a process for making an enzyme preparation, said process comprising the step of mixing components (a), (b), and (c) as disclosed above, wherein component (b) preferably comprises at least one protease selected from the group of serine endopeptidases (EC 3.4.21), most preferably at least one protease selected from the group of subtilisin type proteases (EC 3.4.21.62); and optionally at least one enzyme selected from the group of lipases and amylases. In one embodiment component (c) comprises at least one solvent as disclosed above. In one embodiment, component (c) comprises at least one enzyme stabilizer different from component (a), preferably selected from boron containing compounds as disclosed above, more preferably selected from phenyl boronic acid (PBA) or its derivatives as disclosed above, most preferably being 4-formyl phenyl boronic acid (4-FPBA) all as disclosed above.

Component (b) may be solid. Solid component (b) may be added to solid component (a) prior to contact of both with at least one solvent (component (c)). At least one solvent is as disclosed above. Contact with at least one solvent (component (c)) may result in solubilizing of at least one molecule component (a) and at least one molecule component (b), resulting in stabilization of at least one molecule component (b). In one embodiment, solid components (a) and (b) are completely dissolved in at least one solvent (component (c)) without phase separation. Solid component (a) may be dissolved in at least one solvent (component (c)) prior to mixing with solid or liquid component (b). In one embodiment, component (a) is completely dissolved in at least one solvent (component (c)) prior to mixing with component (b). At least one solvent is as disclosed above.

Component (b) may be liquid, wherein at least one enzyme may be comprised in a liquid enzyme concentrate as disclosed above. Liquid component (b) may be supplemented with solid component (a), wherein solid component (a) dissolves in liquid component (b). In one embodiment, liquid component (b) is aqueous, preferably resulting from fermentation. In one embodiment, when solid component (a) dissolves in liquid component (b), no additional solvent may be added.

In one embodiment, component (c) as disclosed above is mixed with components (a) and (b), wherein the mixing is characterized in being done in one or more steps.

### Enzyme Stabilization

The invention relates to a method of stabilizing component (b) by the step of adding component (a), wherein components (a) and (b) are those disclosed above. In one embodiment, component (b) is liquid. In one embodiment, the invention relates to a method of stabilizing component (b) by the step of adding component (a), wherein component (b) comprises at least one protease and optionally an enzyme selected from lipase and amylase.

In one embodiment, the invention relates to a method of stabilizing component (b) by the step of adding component (a) and at least one enzyme stabilizer different from component (a) as disclosed above. At least one enzyme stabilizer different from component (a) is preferably selected from boron containing compounds as disclosed above, more preferably selected from phenyl boronic acid (PBA) or its derivatives as disclosed above, most preferably being 4-formyl phenyl boronic acid (4-FPBA).

The invention further relates to a method of stabilizing at least one hydrolase in liquid formulations comprising the mixing in no specified order in one or more steps at least components (a) and (b) and optionally at least one enzyme stabilizer different from component (a) all as disclosed above with one or more formulation components. At least one enzyme stabilizer different from component (a) is preferably selected from boron containing compounds as disclosed above, more preferably selected from phenyl boronic acid (PBA) or its derivatives as disclosed above, most preferably being 4-formyl phenyl boronic acid (4-FPBA).

In one embodiment, the invention relates to a method of stabilizing component (b) in the presence of at least one surfactant by the step of adding component (a) and optionally at least one enzyme stabilizer different from component (a), wherein components (a) and (b) are those disclosed above and at least one surfactant is selected from non-ionic surfactants, amphoteric surfactants, anionic surfactants, and cationic surfactants, all as described below. In one embodiment, liquid formulations are detergent formulations. At least one enzyme stabilizer different from component (a) is preferably selected from boron containing compounds as disclosed above, more preferably selected from phenyl boronic acid (PBA) or its derivatives as disclosed above, most preferably being 4-formyl phenyl boronic acid (4-FPBA).

The invention relates to the use of component (a) as additive for component (b). In one embodiment, components (a) and (b) are solid, and component (b) is stabilized when contacting the mixture of the solid components (a) and (b) with at least one solvent (component (c) as disclosed above). Contact with at least one solvent (component (c)) may result in solubilizing of at least one molecule component (a) and at least one molecule component (b), resulting in stabilization of at least one molecule component (b). In one embodiment, solid components (a) and (b) are completely dissolved in at least one solvent (component (c)) without phase separation.

In one embodiment, the invention relates to the use of a compound according to formula (I):

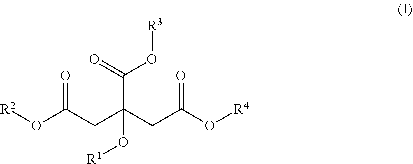

wherein the variables in formula (I) are defined as follows:
R¹ is selected from H and C₁-C₁₀ alkylcarbonyl, wherein alkyl may be linear or branched and may bear one or more hydroxyl groups;
R², R³, R⁴ are independently from each other selected from H, linear C₁-C₈ alkyl, and branched C₃-C₈ alkyl, C₆-C₁₀-aryl, non-substituted or substituted with one or more carboxylate or hydroxyl groups, and C₆-C₁₀-aryl-alkyl, wherein alkyl of the latter is selected from linear C₁-C₈ alkyl or branched C₃-C₈ alkyl, wherein at least one of R², R³, and R⁴ is not H as additive for at least one hydrolase (component (b)), wherein the compound according to formula (I) and the hydrolase are solid, and wherein enzymatic activity of the hydrolase is stabilized when the compound according to formula (I) and the hydrolase are contacted with at least one solvent [component (c)].

In one embodiment of the present invention, component (a) is added in amounts in the range of 0.1% to 30% by weight, relative to the total weight of the enzyme preparation. The enzyme preparation may comprise component (a) in amounts in the range of 0.1% to 15% by weight, 0.25% to 10% by weight, 0.5% to 10% by weight, 0.5% to 6% by weight, or 1% to 3% by weight, all relative to the total weight of the enzyme preparation.

In one embodiment, at least one enzyme stabilizer different from component (a) is added in amounts effective to reversibly inhibit the proteolytic activity of at least one protease comprised in component (b).

In one embodiment, said compound according to formula (I) is used as an additive for component (b), wherein component (b) comprises at least one protease selected from the group of serine endopeptidases (EC 3.4.21), most preferably at least one enzyme selected from the group of subtilisin type proteases (EC 3.4.21.62), wherein the compound according to formula (I) and the protease are solid, and wherein proteolytic activity of the protease is stabilized when the compound according to formula (I) and the protease are contacted with at least one solvent [component (c)].

In one embodiment, said compound according to formula (I) is used together with at least one enzyme stabilizer different from component (a), preferably at least one boron containing compound, as additive for component (b), wherein component (b) comprises at least one protease selected from the group of serine endopeptidases (EC 3.4.21), most preferably at least one enzyme selected from the group of subtilisin type proteases (EC 3.4.21.62), wherein the compound according to formula (I) and the protease are solid, and wherein proteolytic activity of the protease is stabilized when the compound according to formula (I) and the protease are contacted with at least one solvent [component (c)].

In one embodiment, component (b) comprises at least one protease selected from the group of serine endopeptidases (EC 3.4.21), preferably selected from the group of subtilisin type proteases (EC 3.4.21.62), and at least one triacylglycerol lipase according to amino acids 1-269 of SEQ ID NO:2 of U.S. Pat. No. 5,869,438 or a variant thereof having lipolytic activity as disclosed above, wherein the compound according to formula (I) the protease, and the lipase are solid, and wherein proteolytic activity of the protease and/or lipolytic activity of the lipase are stabilized when the compound according to formula (I), the protease, and the lipase are contacted with at least one solvent [component (c)].

In one embodiment, component (b) comprises at least one protease selected from proteases according to SEQ ID NO:22 as described in EP 1921147 or variants thereof having proteolytic activity as disclosed above, and at least one triacylglycerol lipase according to amino acids 1-269 of SEQ ID NO:2 of U.S. Pat. No. 5,869,438 or a variant thereof having lipolytic activity as disclosed above, wherein the compound according to formula (I), the protease, and the lipase are solid, and wherein proteolytic activity of the protease and/or lipolytic activity of the lipase are stabilized when the compound according to formula (I), the protease, and the lipase are contacted with at least one solvent [component (c)].

In one embodiment, component (b) comprises at least one proteases selected from subtilisin 309 as disclosed in Table I a) of WO 89/06279 or variants thereof having proteolytic activity as disclosed above, and at least one triacylglycerol lipase according to amino acids 1-269 of SEQ ID NO:2 of U.S. Pat. No. 5,869,438 or a variant thereof having lipolytic activity as disclosed above, wherein the compound according to formula (I), the protease, and the lipase are solid, and wherein proteolytic activity of the protease and/or lipolytic activity of the lipase are stabilized when the compound according to formula (I), the protease, and the lipase are contacted with at least one solvent [component (c)].

Stabilization of an enzyme may relate to stability in the course of time (e.g. storage stability), thermal stability, pH stability, and chemical stability. The term “enzyme stability” herein preferably relates to the retention of enzymatic activity as a function of time e.g. during storage or operation. The term “storage” herein means to indicate the fact of products or compositions being stored from the time of being manufactured to the point in time of being used in final application. Retention of enzymatic activity as a function of time during storage is called “storage stability”. In one embodiment, storage means storage for at least 20 days at 37° C. Storage may mean storage for 21, 28, or 35 days at 37° C.

To determine changes in enzymatic activity over time, the “initial enzymatic activity” of an enzyme may be measured under defined conditions at time zero (i.e. before storage) and the “enzymatic activity after storage” may be measured at a certain point in time later (i.e. after storage)

The enzymatic activity after storage divided by the initial enzymatic activity multiplied by 100 gives the “residual enzymatic activity” (a %).

An enzyme is stable according to the invention, when its residual enzymatic activity is at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5% or 100% when compared to the initial enzymatic activity before storage.

Subtracting a % from 100% gives the “loss of enzymatic activity during storage” when compared to the initial enzymatic activity before storage. In one embodiment, an enzyme is stable according to the invention when essentially no loss of enzymatic activity occurs during storage, i.e. loss in enzymatic activity equals 0% when compared to the initial enzymatic activity before storage. Essentially no loss of enzymatic activity within this invention may mean that the loss of enzymatic activity is less than 30%, less than 25%, less than 20%, less than 15%, less than 10%, less than 9%, less than 8%, less than 7%, less than 6%, less than 5%, less than 4%, less than 3%, less than 2%, or less than 1% when compared to the initial enzymatic activity before storage.

In one aspect of the invention component (a) is used to reduce loss of enzymatic activity during storage of component (b). Calculation of % reduced loss of enzymatic activity is done as follows: (% loss of enzymatic activity of stabilized enzyme)−(% loss of enzymatic activity of non-stabilized enzyme). The value for reduced loss indicates the reduced loss of enzymatic activity of at least one enzyme comprised in component (b) in the presence of component (a) when compared to the loss of enzymatic activity of the same enzyme(s) in the absence of component (a) at a certain point in time.

Reduced loss of enzymatic activity within this invention may mean that the loss of enzymatic activity is reduced in the presence of component (a) by at least 5%, by at least 10%, by at least 15%, by at least 20%, by at least 25%, by at least 30%, by at least 40%, by at least 50%, by least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5%, when compared to the loss of enzymatic activity in the absence of component (a).

In one aspect, the invention relates to a method of reducing loss of proteolytic activity of at least one protease (component (b)) comprised in a liquid during storage by the step of adding a compound according to formula (I):

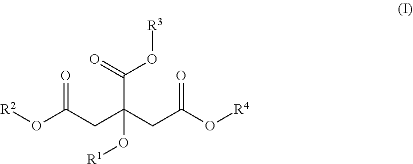

wherein the variables in formula (I) are defined as follows:

R¹ is selected from H and C₁-C₁₀ alkylcarbonyl, wherein alkyl may be linear or branched and may bear one or more hydroxyl groups;

R², R³, R⁴ are independently from each other selected from H, linear C₁-C₈ alkyl, and branched C₃-C₈ alkyl, C₆-C₁₀-aryl, non-substituted or substituted with one or more carboxylate or hydroxyl groups, and C₆-C₁₀-aryl-alkyl, wherein alkyl of the latter is selected from linear C₁-C₈ alkyl or branched C₃-C₈ alkyl, wherein at least one of R², R³, and R⁴ is not H.

In one embodiment, the method of reducing loss of proteolytic activity of at least one protease (component (b)) comprised in a liquid during storage comprises the step of adding a compound according to formula (I) and the step of adding at least one enzyme stabilizer different from component (a), preferably a boron containing compound.

In one embodiment, the protease (component (b)) is comprised in a liquid enzyme preparation, or the protease is comprised in a liquid composition comprising at least one surfactant such as a liquid detergent formulation.

In one embodiment, the method of reducing loss of proteolytic activity of at least one protease, is characterized in component (b) comprising at least one protease selected from the group of serine endopeptidases (EC 3.4.21), most preferably at least one enzyme selected from the group of subtilisin type proteases (EC 3.4.21.62).

In one embodiment, component (b) comprises at least one protease selected from the group of serine endopeptidases (EC 3.4.21), preferably selected from the group of subtilisin type proteases (EC 3.4.21.62), and at least one triacylglycerol lipase according to amino acids 1-269 of SEQ ID NO:2 of U.S. Pat. No. 5,869,438 or a variant thereof having lipolytic activity as disclosed above. In one embodiment, component (b) comprises at least one protease selected from proteases according to SEQ ID NO:22 as described in EP 1921147 or variants thereof having proteolytic activity as disclosed above, and at least one triacylglycerol lipase according to amino acids 1-269 of SEQ ID NO:2 of U.S. Pat. No. 5,869,438 or a variant thereof having lipolytic activity as disclosed above.

In one embodiment, component (b) comprises at least one proteases selected from subtilisin 309 as disclosed in Table I a) of WO 89/06279 or variants thereof having proteolytic activity as disclosed above, and at least one triacylglycerol lipase according to amino acids 1-269 of SEQ ID NO:2 of U.S. Pat. No. 5,869,438 or a variant thereof having lipolytic activity as disclosed above.

In one embodiment, component (b) comprises at least one protease selected from the group of serine endopeptidases (EC 3.4.21), preferably selected from the group of subtilisin type proteases (EC 3.4.21.62), and at least one amylase, preferably selected from SEQ ID NO: 1 and SEQ ID NO: 2 of WO 2013/001078 and variants thereof having amylolytic activity.

In one embodiment, component (b) comprises at least one protease selected from proteases according to SEQ ID NO:22 as described in EP 1921147 or variants thereof having proteolytic activity as disclosed above, and at least one amylase, preferably selected from SEQ ID NO: 1 and SEQ ID NO: 2 of WO 2013/001078 and variants thereof having amylolytic activity.

In one embodiment, component (b) comprises at least one proteases selected from subtilisin 309 as disclosed in Table I a) of WO 89/06279 or variants thereof having proteolytic activity as disclosed above, and at least one amylase, preferably at least one amylase is selected from amylases from Bacillus sp. 707 or variants thereof having amylolytic activity and amylases from Bacillus halmapalus or variants thereof having amylolytic activity, preferably selected from amylases according to SEQ ID NO: 1 and 2 of WO 2013/001078 or variants thereof having amylolytic activity—all as disclosed above.

In one aspect of the invention, component (a) stabilizes at least one protease comprised in component (b). At least one protease comprised in component (b) is preferably selected from subtilisin 147 and/or 309 as disclosed in WO 89/06279 and variants thereof having proteolytic activity, subtilisin from Bacillus lentus as disclosed in WO 91/02792 and variants thereof having proteolytic activity, and subtilisin according to SEQ ID NO:22 as described in EP 1921147 and variants thereof having proteolytic activity—all as disclosed above. In one embodiment, component (a) is used to stabilize protease [component (b)] within a liquid enzyme preparation. In one embodiment, component (a) is used to stabilize protease [component (b)] within a liquid composition comprising at least one surfactant, preferably within a liquid detergent composition. Stabilization in this context may mean stabilization during storage at 37° C. for 21, 28 and/or 35 days. In one embodiment, the addition of component (a) to component (b) stabilizes protease during storage, wherein stabilization is characterized by
(a) residual proteolytic activity after storage at 37° C. for 21 days being >70%, ≥75%, or ≥80% when compared to the initial proteolytic activity before storage and/or
(b) residual proteolytic activity after storage at 37° C. for 28 days being >65%, ≥70%, or ≥75% when compared to the initial proteolytic activity before storage and/or
(c) residual proteolytic activity after storage at 37° C. for 35 days being >60%, or ≥65% when compared to the initial proteolytic activity before storage.

In one embodiment, the addition of component (a) to component (b) stabilizes protease during storage, wherein component (a) is characterized by R¹ in the compound according to formula (I) is H, and R², R³, R⁴ are selected from linear C₂-C₄ alkyl, and wherein stabilization is characterized by
(a) residual proteolytic activity after storage at 37° C. for 21 days being >70%, ≥75%, ≥80%, or ≥83% when compared to the initial proteolytic activity before storage and/or
(b) residual proteolytic activity after storage at 37° C. for 28 days being >65%, ≥70%, ≥75%, or ≥79% when compared to the initial proteolytic activity before storage and/or
(c) residual proteolytic activity after storage at 37° C. for 35 days being >60%, or ≥65%, ≥70%, or ≥72% when compared to the initial proteolytic activity before storage.

In one embodiment, the addition of component (a) to component (b) stabilizes protease during storage, wherein component (a) is characterized by R¹ in the compound according to formula (I) is acetyl, and R², R³, R⁴ are selected from linear C₂-C₄ alkyl, preferably C₂ and C₄ alkyl, and wherein stabilization is characterized by
(a) residual proteolytic activity after storage at 37° C. for 21 days being >70%, ≥75%, ≥80%, or ≥85% when compared to the initial proteolytic activity before storage and/or
(b) residual proteolytic activity after storage at 37° C. for 28 days being >65%, ≥70%, ≥75%, ≥80%, or ≥81% when compared to the initial proteolytic activity before storage and/or
(c) residual proteolytic activity after storage at 37° C. for 35 days being >60%, or ≥65%, ≥70%, ≥75%, or ≥77% when compared to the initial proteolytic activity before storage.

In one embodiment, the addition of component (a) to component (b) stabilizes protease during storage, wherein component (a) is characterized by R¹ and R² in the compound according to formula (I) are H, R⁴ is selected from linear C₂-C₄ alkyl, preferably C₂ alkyl, and R³ equals either R¹/R² or R⁴, and wherein stabilization is characterized by
(a) residual proteolytic activity after storage at 37° C. for 21 days being >70%, ≥75%, ≥80%, or ≥81% when compared to the initial proteolytic activity before storage and/or
(b) residual proteolytic activity after storage at 37° C. for 28 days being >65%, ≥70%, ≥75%, or ≥76% when compared to the initial proteolytic activity before storage and/or
(c) residual proteolytic activity after storage at 37° C. for 35 days being >60%, ≥65%, or ≥69% when compared to the initial proteolytic activity before storage.

In one embodiment, the addition of component (a) to component (b) stabilizes protease during storage, wherein component (a) is characterized by R¹ in the compound according to formula (I) is H, and R², R³, R⁴ are selected from phenylmethyl, and salicyl, and wherein stabilization is characterized by
(a) residual proteolytic activity after storage at 37° C. for 21 days being >70%, ≥75%, ≥80%, or ≥85% when compared to the initial proteolytic activity before storage and/or
(b) residual proteolytic activity after storage at 37° C. for 28 days being >65%, ≥70%, ≥75%, ≥80%, or ≥82% when compared to the initial proteolytic activity before storage and/or
(c) residual proteolytic activity after storage at 37° C. for 35 days being >60%, ≥65%, ≥70%, or ≥75% when compared to the initial proteolytic activity before storage.

In one embodiment, the addition of component (a) to component (b) stabilizes protease during storage, wherein stabilization is characterized by
(a) loss of proteolytic activity during storage at 37° C. for 21 days being <25%, or ≥20% when compared to the initial proteolytic activity before storage and/or
(b) loss of proteolytic activity during storage at 37° C. for 28 days being <30%, or ≥25% when compared to the initial proteolytic activity before storage and/or
(c) loss of proteolytic activity during storage at 37° C. for 35 days being <40%, or ≥35% when compared to the initial proteolytic activity before storage.

In one embodiment, the addition of component (a) to component (b) stabilizes protease during storage, wherein component (a) is characterized by R¹ in the compound according to formula (I) is H, and R², R³, R⁴ are selected from linear C₂-C₄ alkyl, and wherein stabilization is characterized by
(a) loss of proteolytic activity during storage at 37° C. for 21 days being <25%, ≥20%, or ≥17% when compared to the initial proteolytic activity before storage and/or
(b) loss of proteolytic activity during storage at 37° C. for 28 days being <30%, ≥25%, or ≥22% when compared to the initial proteolytic activity before storage.
(c) loss of proteolytic activity during storage at 37° C. for 35 days being <40%, or ≥35%, ≥30%, or ≥28% when compared to the initial proteolytic activity before storage.

In one embodiment, the addition of component (a) to component (b) stabilizes protease during storage, wherein component (a) is characterized by R¹ in the compound according to formula (I) is acetyl, and R², R³, R⁴ are selected from linear C₂-C₄ alkyl, preferably C₂ and C₄ alkyl, and wherein stabilization is characterized by
(a) loss of proteolytic activity during storage at 37° C. for 21 days being <25%, ≥20%, or ≥17% when compared to the initial proteolytic activity before storage and/or
(b) loss of proteolytic activity during storage at 37° C. for 28 days being <30%, ≥25%, or ≥21% when compared to the initial proteolytic activity before storage.
(c) loss of proteolytic activity during storage at 37° C. for 35 days being <40%, ≥35%, ≥30%, or ≥25% when compared to the initial proteolytic activity before storage.

In one embodiment, the addition of component (a) to component (b) stabilizes protease during storage, wherein component (a) is characterized by R¹ and R² in the compound according to formula (I) are H, R⁴ is selected from linear C₂-C₄ alkyl, preferably C₂ alkyl, and R³ equals either R¹/R² or R⁴, and wherein stabilization is characterized by
(a) loss of proteolytic activity during storage at 37° C. for 21 days being <25%, ≥20%, or ≥19% when compared to the initial proteolytic activity before storage and/or
(b) loss of proteolytic activity during storage at 37° C. for 28 days being <30%, ≥25%, or ≥24% when compared to the initial proteolytic activity before storage.
(c) loss of proteolytic activity during storage at 37° C. for 35 days being <40%, ≥35%, ≥30%, or ≥31% when compared to the initial proteolytic activity before storage.

In one embodiment, the addition of component (a) to component (b) stabilizes protease during storage, wherein component (a) is characterized by R¹ in the compound according to formula (I) is H, and R², R³, R⁴ are selected from phenylmethyl, and salicyl, and wherein stabilization is characterized by
(a) loss of proteolytic activity during storage at 37° C. for 21 days being <25%, ≥20%, or ≥15% when compared to the initial proteolytic activity before storage and/or
(b) loss of proteolytic activity during storage at 37° C. for 28 days being <30%, ≥25%, ≥20%, or ≥17% when compared to the initial proteolytic activity before storage.
(c) loss of proteolytic activity during storage at 37° C. for 35 days being <40%, ≥35%, ≥30%, ≥25%, or ≥23% when compared to the initial proteolytic activity before storage.

In one embodiment, the addition of component (a) to component (b) stabilizes protease during storage, wherein stabilization is characterized by
(a) reduced loss of proteolytic activity during storage at 37° C. for 21 days being ≥10% when compared to the loss of proteolytic activity in the absence of component (a) and/or
(b) reduced loss of proteolytic activity during storage at 37° C. for 28 days being ≥10% when compared to the loss of proteolytic activity in the absence of component (a) and/or
(c) reduced loss of proteolytic activity during storage at 37° C. for 35 days being ≥10% when compared to the loss of proteolytic activity in the absence of component (a).

In one embodiment, the addition of component (a) to component (b) stabilizes protease during storage, wherein component (a) is characterized by R¹ in the compound according to formula (I) is H, and R², R³, R⁴ are selected from linear C₂-C₄ alkyl, and wherein stabilization is characterized by
(a) reduced loss of proteolytic activity during storage at 37° C. for 21 days being ≥10%, or ≥11% when compared to the loss of proteolytic activity in the absence of component (a) and/or
(b) reduced loss of proteolytic activity during storage at 37° C. for 28 days being ≥10%, or ≥14% when compared to the loss of proteolytic activity in the absence of component (a) and/or
(c) reduced loss of proteolytic activity during storage at 37° C. for 35 days being ≥10%, or ≥12% when compared to the loss of proteolytic activity in the absence of component (a).

In one embodiment, the addition of component (a) to component (b) stabilizes protease during storage, wherein component (a) is characterized by R¹ in the compound according to formula (I) is acetyl, and R², R³, R⁴ are selected from linear C₂-C₄ alkyl, preferably C₂ and C₄ alkyl, and wherein stabilization is characterized by
(a) reduced loss of proteolytic activity during storage at 37° C. for 21 days being ≥10%, or ≥14% when compared to the loss of proteolytic activity in the absence of component (a) and/or
(b) reduced loss of proteolytic activity during storage at 37° C. for 28 days being ≥10%, or ≥15% when compared to the loss of proteolytic activity in the absence of component (a) and/or
(c) reduced loss of proteolytic activity during storage at 37° C. for 35 days being ≥10%, or 15% when compared to the loss of proteolytic activity in the absence of component (a).

In one embodiment, the addition of component (a) to component (b) stabilizes protease during storage, wherein component (a) is characterized by R¹ and R² in the compound according to formula (I) are H, R⁴ is selected from linear C₂-C₄ alkyl, preferably C₂ alkyl, and R³ equals either R¹/R² or R⁴, and wherein stabilization is characterized by
(a) reduced loss of proteolytic activity during storage at 37° C. for 21 days being ≥10%, or ≥12% when compared to the loss of proteolytic activity in the absence of component (a) and/or
(b) reduced loss of proteolytic activity during storage at 37° C. for 28 days being ≥10% when compared to the loss of proteolytic activity in the absence of component (a) and/or (c) reduced loss of proteolytic activity during storage at 37° C. for 35 days being ≥10% when compared to the loss of proteolytic activity in the absence of component (a).

In one embodiment, the addition of component (a) to component (b) stabilizes protease during storage, wherein component (a) is characterized by R¹ in the compound according to formula (I) is H, and R², R³, R⁴ are selected from phenylmethyl, and salicyl, and wherein stabilization is characterized by
(a) reduced loss of proteolytic activity during storage at 37° C. for 21 days being ≥10%, or ≥13% when compared to the loss of proteolytic activity in the absence of component (a) and/or
(b) reduced loss of proteolytic activity during storage at 37° C. for 28 days being ≥10%, ≥15%, or ≥16% when compared to the loss of proteolytic activity in the absence of component (a) and/or
(c) reduced loss of proteolytic activity during storage at 37° C. for 35 days being ≥10%, ≥15%, ≥20%, ≥25%, or ≥29% when compared to the loss of proteolytic activity in the absence of component (a).

In embodiments of the above embodiments, component (a) is used to stabilize protease [component (b)] within a liquid enzyme preparation. Further, in embodiments of the above embodiments the protease which is stabilized by component (a) is selected from subtilisin 147 and/or 309 as disclosed in WO 89/06279 and variants thereof having proteolytic activity, subtilisin from Bacillus lentus as disclosed in WO 91/02792 and variants thereof having proteolytic activity, and subtilisin according to SEQ ID NO:22 as described in EP 1921147 and variants thereof having proteolytic activity—all as disclosed above.

In one aspect of the invention, component (a) is used to stabilize component (b) comprising at least one protease and at least one lipase within a liquid composition comprising at least one surfactant, preferably within a liquid detergent composition, wherein
at least one protease is preferably selected from subtilisin 147 and/or 309 as disclosed in WO 89/06279 and variants thereof having proteolytic activity, subtilisin from Bacillus lentus as disclosed in WO 91/02792 and variants thereof having proteolytic activity, and subtilisin according to SEQ ID NO:22 as described in EP 1921147 and variants thereof having proteolytic activity—all as disclosed above;
at least one lipase is selected from Thermomyces lanuginosa lipase and variants thereof, preferably triacylglycerol lipase according to amino acids 1-269 of SEQ ID NO:2 of U.S. Pat. No. 5,869,438 or a variant thereof having lipolytic activity—all as disclosed above.

In one aspect of the invention, component (a) is used to stabilize component (b) comprising at least one protease and at least one amylase within a liquid composition comprising at least one surfactant, preferably within a liquid detergent composition, wherein
at least one protease is preferably selected from subtilisin 147 and/or 309 as disclosed in WO 89/06279 and variants thereof having proteolytic activity, subtilisin from Bacillus lentus as disclosed in WO 91/02792 and variants thereof having proteolytic activity, and subtilisin according to SEQ ID NO:22 as described in EP 1921147 and variants thereof having proteolytic activity—all as disclosed above;
at least one amylase is selected from amylases from Bacillus sp. 707 or variants thereof having amylolytic activity and amylases from Bacillus halmapalus or variants thereof having amylolytic activity, preferably selected from amylases according to SEQ ID NO: 1 and 2 of WO 2013/001078 or variants thereof having amylolytic activity—all as disclosed above.

### Use of Enzyme Preparation for Formulation Processes

The invention in one aspect relates to the use of the liquid enzyme preparation of the invention to be formulated into detergent formulations such as I&I and homecare formulations for laundry and hard surface cleaning, wherein at least components (a) and (b) are mixed in no specified order in one or more steps with one or more detergent components. In one embodiment, at least components (a), (b) and (c) are mixed in no specified order in one or more steps with one or more detergent components, wherein component (c) comprises at least one enzyme stabilizer different from component (a), preferably a boron-containing compound.

In one aspect of the invention relates to a detergent formulation comprising the liquid enzyme preparation of the invention and one or more detergent components.

Detergent components vary in type and/or amount in a detergent formulation depending on the desired application such as laundering white textiles, colored textiles, and wool. The component(s) chosen further depend on physical form of a detergent formulation (liquid, solid, gel, provided in pouches or as a tablet, etc). The component(s) chosen e.g. for laundering formulations further depend on regional conventions which themselves are related to aspects like washing temperatures used, mechanics of laundry machine (vertical vs. horizontal axis machines), water consumption per wash cycle etc. and geographical characteristics like average hardness of water.

Individual detergent components and usage in detergent formulations are known to those skilled in the art. Suitable detergent components comprise inter alia surfactants, builders, polymers, alkaline, bleaching systems, fluorescent whitening agents, suds suppressors and stabilizers, hydrotropes, and corrosion inhibitors. Further examples are described e.g. in “complete Technology Book on Detergents with Formulations (Detergent Cake, Dishwashing Detergents, Liquid & Paste Detergents, Enzyme Detergents, Cleaning Powder & Spray Dried Washing Powder)”, Engineers India Research Institute (EIRI), 6th edition (2015). Another reference book for those skilled in the art may be “Detergent Formulations Encyclopedia”, Solverchem Publications, 2016.

It is understood that the detergent components are in addition to the components comprised in the enzyme preparation of the invention. If a component comprised in the enzyme preparation of the invention is also a detergent component, it might be the concentrations that need to be adjusted that the component is effective for the purpose desired in the detergent formulation. Detergent components may have more than one function in the final application of a detergent formulation, therefore any detergent component mentioned in the context of a specific function herein, may also have another function in the final application of a detergent formulation. The function of a specific detergent component in the final application of a detergent formulation usually depends on its amount within the detergent formulation, i.e. the effective amount of a detergent component.

The term “effective amount” includes amounts of individual components to provide effective stain removal and/or effective cleaning conditions (e.g. pH, quantity of foaming), amounts of certain components to effectively provide optical benefits (e.g. optical brightening, dye transfer inhibition), and/or amounts of certain components to effectively aid the processing (maintain physical characteristics during processing, storage and use; e.g. viscosity modifiers, hydrotropes, desiccants).

In one embodiment, a detergent formulation is a formulation of more than two detergent components, wherein at least one component is effective in stain-removal, at least one component is effective in providing the optimal cleaning conditions, and at least one component is effective in maintaining the physical characteristics of the detergent.

Detergent formulations of the invention may comprise component (a) and component (b) being dissolved in solvent. Dissolved may mean being dissolved in the overall detergent formulation. Dissolved may mean component (a) and component (b) being part of the liquid enzyme preparation of the invention which may be encapsulated. Encapsulated liquid enzyme preparation may be part of a liquid detergent formulation or part of a solid detergent formulation.

In one embodiment of the present invention, detergent formulations, preferably liquid detergent formulations, comprise component (a) in amounts in the range of 0.1% to 30% by weight, relative to the total weight of the detergent formulation. The enzyme preparation may comprise component (a) in amounts in the range of 0.1% to 15% by weight, 0.25% to 10% by weight, 0.5% to 10% by weight, 0.5% to 6% by weight, or 1% to 3% by weight, all relative to the total weight of the detergent formulation.

In one embodiment of the present invention, detergent formulations, preferably liquid detergent formulations, comprise 0.5 to 20% by weight, particularly 1-10% by weigh component (b) and 0.01% to 10% of component (a), more particularly 0.05 to 5% by weight and most particularly 0.1% to 2% by weight of component (a), all relative to the total weight of the detergent formulation.

Detergent formulations of the invention comprise at least one compound selected from surfactants, builders, polymers, fragrances and dyestuffs.

The detergent formulation of the invention comprises at least one surfactant selected from non-ionic surfactants, amphoteric surfactants, anionic surfactants, and cationic surfactants.

The detergent formulation may comprise 0.1 to 60% by weight relative to the total weight of the detergent formulation of surfactant. The detergent formulation may comprise at least one compound selected from anionic surfactants, non-ionic surfactants, amphoteric surfactants, and amine oxide surfactants as well as combinations of at least two of the foregoing. In one embodiment, the detergent formulation of the invention comprises 5 to 30% by weight of anionic surfactant and at least one non-ionic surfactant, for example in the range of from 3 to 20% by weight, all relative to the total weight of the detergent formulation, wherein the detergent formulation may be liquid.

At least one non-ionic surfactant may be selected from alkoxylated alcohols, di- and multiblock copolymers of ethylene oxide and propylene oxide and reaction products of sorbitan with ethylene oxide or propylene oxide, alkyl polyglycosides (APG), hydroxyalkyl mixed ethers and amine oxides.

Preferred examples of alkoxylated alcohols and alkoxylated fatty alcohols are, for example, compounds of the general formula (IV)

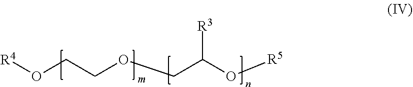

wherein
R³ is identical or different and selected from hydrogen and linear C₁-C₁₀-alkyl, preferably in each case identical and ethyl and particularly preferably hydrogen or methyl,
R⁴ is selected from C₈-C₂₂-alkyl, branched or linear, for example n-C₈H₁₇, n-C₁₀H₂₁, n-C₁₂H₂₅, n-C₁₄H₂₉, n-C₁₆H₃₃ or n-C₁₈H₃₇,
R⁵ is selected from C₁-C₁₀-alkyl, methyl, ethyl, n-propyl, isopropyl, n-butyl, isobutyl, sec-butyl, tert-butyl, n-pentyl, isopentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl or isodecyl.

The variables m and n are in the range from zero to 300, where the sum of n and m is at least one, preferably in the range of from 3 to 50. Preferably, m is in the range from 1 to 100 and n is in the range from 0 to 30.

In one embodiment, compounds of the general formula (IV) may be block copolymers or random copolymers, preference being given to block copolymers.

Other preferred examples of alkoxylated alcohols are, for example, compounds of the general formula (V):

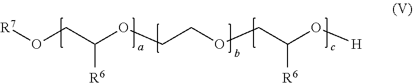

wherein
R⁶ is identical or different and selected from hydrogen and linear C₁-C₁₀-alkyl, preferably identical in each case and ethyl and particularly preferably hydrogen or methyl,
R⁷ is selected from C₆-C₂₀-alkyl, branched or linear, in particular n-C₈H₁₇, n-C₁₀H₂₁, n-C₁₂H₂₅, n-C₁₃H₂₇, n-C₁₅H₃₁, n-C₁₄H₂₉, n-C₁₆H₃₃, n-C₁₈H₃₇,
a is a number in the range from zero to 10, preferably from 1 to 6,
b is a number in the range from 1 to 80, preferably from 4 to 20,
c is a number in the range from zero to 50, preferably 4 to 25.

The sum a+b+c is preferably in the range of from 5 to 100, even more preferably in the range of from 9 to 50.

In one embodiment, an alkoxylated alcohol is selected from those according to formula (V), wherein there is no R⁶ and R⁷ is selected from n-C₈H₁₇, n-C₁₀H₂₁, n-C₁₂H₂₅, n-C₁₃H₂₇, n-C₁₅H₃₁, n-C₁₄H₂₉, n-C₁₆H₃₃, n-C₁₈H₃₇; a and c are zero, b is in the range from 4 to 20, preferably 9.

Preferred examples for hydroxyalkyl mixed ethers are compounds of the general formula (VI)

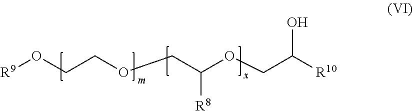

in which the variables are defined as follows:
R⁸ is identical or different and selected from hydrogen and linear C₁-C₁₀-alkyl, preferably in each case identical and ethyl, and particularly preferably hydrogen or methyl,
R⁹ is selected from linear or branched C₈-C₂₂-alkyl and C₈-C₂₂-alkenyl; example include iso-C₁₁H₂₃, iso-C₁₃H₂₇, n-C₈H₁₇, n-C₁₀H₂₁, n-C₁₂H₂₅, n-C₁₄H₂₉, n-C₁₆H₃₃ or n-C₁₈H₃₇,
R¹⁰ is selected from linear or branched C₁-C₁₈-alkyl and C₂-C₁₈ alkenyl; examples include methyl, ethyl, n-propyl, isopropyl, n-butyl, isobutyl, sec-butyl, tert-butyl, n-pentyl, iso-pentyl, sec-pentyl, neopentyl, 1,2-dimethylpropyl, isoamyl, n-hexyl, isohexyl, sec-hexyl, n-heptyl, n-octyl, 2-ethylhexyl, n-nonyl, n-decyl, isodecyl, n-dodecyl, n-tetradecyl, n-hexadecyl, and n-octadecyl.

The variables m and x are in the range from zero to 300, preferably in the range from zero to 100; the sum of m and x is at least one, preferably in the range of from 5 to 50.

Compounds of the general formulae (V) and (VI) may be block copolymers or random copolymers, preference being given to block copolymers.

Further suitable nonionic surfactants are selected from di- and multiblock copolymers, composed of ethylene oxide and propylene oxide. Further suitable nonionic surfactants are selected from ethoxylated or propoxylated sorbitan esters. Amine oxides or alkyl polyglycosides, especially linear C₄-C₁₈-alkyl polyglucosides and branched C₈-C₁₈-alkyl polyglycosides such as compounds of general average formula (VII) are likewise suitable.

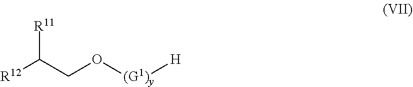

wherein:

R¹¹ is C₁-C₄-alkyl, in particular ethyl, n-propyl or isopropyl,

R¹² is —(CH₂)₂—R¹¹,

G¹ is selected from monosaccharides with 4 to 6 carbon atoms, especially from glucose and xylose,

y in the range of from 1.1 to 4, y being an average number.

Further examples of non-ionic surfactants are compounds of general formula (VIIIa) and (VIIIb)

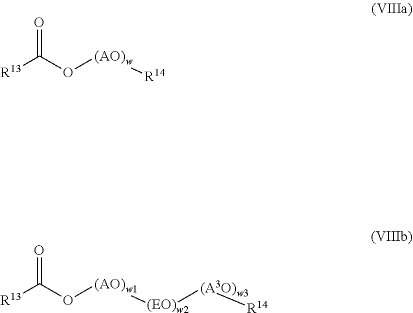

wherein

AO is selected from ethylene oxide, propylene oxide and butylene oxide,

EO is ethylene oxide, CH₂CH₂—O,

R¹³ is C₁-C₄-alkyl, in particular ethyl, n-propyl or isopropyl,

R¹⁴ selected from C₈-C₁₈-alkyl, branched or linear

A³O is selected from propylene oxide and butylene oxide,

w is a number in the range of from 15 to 70, preferably 30 to 50,

w1 and w3 are numbers in the range of from 1 to 5, and

w2 is a number in the range of from 13 to 35.

An overview of suitable further nonionic surfactants can be found in EP-A 0 851 023 and in DEA 198 19 187.

In one embodiment, the detergent formulation comprises mixtures of two or more different nonionic surfactants.

At least one amphoteric surfactant may be selected from surfactants that bear a positive and a negative charge in the same molecule under use conditions. Preferred examples of amphoteric surfactants are so-called betaine-surfactants. Many examples of betaine-surfactants bear one quaternized nitrogen atom and one carboxylic acid group per molecule. A particularly preferred example of amphoteric surfactants is cocamidopropyl betaine (lauramidopropyl betaine).

Examples of amine oxide surfactants are compounds of the general formula (IX)

R¹³R¹⁴R¹⁵N→O (IX)

wherein R¹³, R¹⁴ and R¹⁵ are selected independently from each other from aliphatic, cycloaliphatic or C₂-C₄-alkylene C₁₀-C₂₀-alkylamido moieties. Preferably, R¹² is selected from C₈-C₂₀-alkyl or C₂-C₄-alkylene C₁₀-C₂₀-alkylamido and R¹³ and R¹⁴ are both methyl.

A particularly preferred example is lauryl dimethyl aminoxide, sometimes also called lauramine oxide. A further particularly preferred example is cocamidylpropyl dimethylaminoxide, sometimes also called cocamidopropylamine oxide.

At least one anionic surfactant may be selected from alkali metal and ammonium salts of C₈-C₁₈-alkyl sulfates, of C₈-C₁₈-fatty alcohol polyether sulfates, of sulfuric acid half-esters of ethoxylated C₄-C₁₂-alkylphenols (ethoxylation: 1 to 50 mol of ethylene oxide/mol), C₁₂-C₁₈ sulfo fatty acid alkyl esters, for example of C₁₂-C₁₈ sulfo fatty acid methyl esters, furthermore of C₁₂-C₁₈-alkylsulfonic acids and of C₁₀-C₁₈-alkylarylsulfonic acids. Preference is given to the alkali metal salts of the aforementioned compounds, particularly preferably the sodium salts.

Specific examples of anionic surfactants are compounds according to general formula (X)

CₛH₂ₛ₊₁—O(CH₂CH₂O)ₜ—SO₃M (X)

wherein
s being a number in the range of from 10 to 18, preferably 12 to 14, and even more preferably s=12,
t being a number in the range of from 1 to 5, preferably 2 to 4 and even more preferably 3.
M being selected from alkali metals, preferably potassium and even more preferably sodium.

The variables s and t may be average numbers and therefore they are not necessarily whole numbers, while in individual molecules according to formula (X), both s and t denote whole numbers.

Further examples for suitable anionic surfactants are soaps, for example the sodium or potassium salts of stearic acid, oleic acid, palmitic acid, ether carboxylates, and alkylether phosphates. Inventive detergent formulations may comprise 1 to 40% by weight of at least one detergent builder. Examples for detergent builders include but are not limited to zeolite, phosphate, phosphonate, citrate, polymer builders, or aminocarboxylates such as the alkali metal salts of iminodisuccinates, for example IDS-Na₄, furthermore nitrilotriacetic acid (“NTA”), methylglycine diacetic acid (“MGDA”), glutamic acid diacetic acid (“GLDA”), ethylene diamine tetraacetic acid (“EDTA”) or diethylenetriamine pentaacetic acid (“DTPA”). Preferred alkali metal salts are the potassium salts and especially the sodium salts.

Further examples of detergent builders are polymers with complexing groups like, for example, polyethylenimine in which 20 to 90 mole-% of the N-atoms bear at least one CH₂COO⁻ group, and the respective alkali metal salts of the above sequestrants, especially their sodium salts. Further examples of suitable polymers are polyalkylenimines, for example polyethylenimines and polypropylene imines. Polyalkylenimines may be used as such or as polyalkoxylated derivatives, for examples ethoxylated or propoxylated. Polyalkylenimines comprise at least three alkylenimine units per molecule.

In one embodiment of the present invention, said alkylenimine unit is a C₂-C₁₀-alkylendiamine unit, for example a 1,2-propylendiamine, preferably an α,ω-C₂-C₁₀-alkylendiamine, for example 1,2-ethylendiamine, 1,3-propylendiamine, 1,4-butylendiamine, 1,5-pentylendiaminne, 1,6-hexandiamine (also being referred to as 1,6-hexylendiamine), 1,8-diamine or 1,10-decandiamine, even more preferred are 1,2-ethylendiamine, 1,3-propylendiamine, 1,4-butylendiamine, and 1,6-hexandiamine.

In another embodiment of the present invention, said polyalkylenimine is selected from polyalkylenimine unit, preferably a polyethylenimine or polypropylenimine unit.

The term “polyethylenimine” in the context of the present invention does not only refer to polyethylenimine homopolymers but also to polyalkylenimines comprising NH—CH₂—CH₂—NH structural elements together with other alkylene diamine structural elements, for example NH—CH₂—CH₂—CH₂—NH structural elements, NH—CH₂—CH(CH₃)—NH structural elements, NH—(CH₂)₄—NH structural elements, NH—(CH₂)₆—NH structural elements or (NH—(CH₂)₈—NH structural elements but the NH—CH₂—CH₂—NH structural elements being in the majority with respect to the molar share. Preferred polyethylenimines comprise NH—CH₂—CH₂—NH structural elements being in the majority with respect to the molar share, for example amounting to 60 mol-% or more, more preferably amounting to at least 70 mol-%, referring to all alkylenimine structural elements. In a special embodiment, the term polyethylenimine refers to those polyalkylenimines that bear only one or zero alkylenimine structural element per polyethylenimine unit that is different from NH—CH₂—CH₂—NH.

The term “polypropylenimine” in the context of the present invention does not only refer to polypropylenimine homopolymers but also to polyalkylenimines comprising NH—CH₂—CH(CH₃)—NH structural elements together with other alkylene diamine structural elements, for example NH—CH₂—CH₂—CH₂—NH structural elements, NH—CH₂—CH₂—NH structural elements, NH—(CH₂)₄—NH structural elements, NH—(CH₂)₆—NH structural elements or (NH—(CH₂)₈—NH structural elements but the NH—CH₂—CH(CH₃)—NH structural elements being in the majority with respect to the molar share. Preferred polypropylenimines comprise NH—CH₂—CH(CH₃)—NH structural elements being in the majority with respect to the molar share, for example amounting to 60 mol-% or more, more preferably amounting to at least 70 mol-%, referring to all alkylenimine structural elements. In a special embodiment, the term polypropylenimine refers to those polyalkylenimines that bear only one or zero alkylenimine structural element per polypropylenimine unit that is different from NH—CH₂—CH(CH₃)—NH.

Branches may be alkylenamino groups such as, but not limited to —CH₂—CH₂—NH₂ groups or (CH₂)₃—NH₂-groups. Longer branches may be, for examples, —(CH₂)₃—N(CH₂CH₂CH₂NH₂)₂ or —(CH₂)₂—N(CH₂CH₂NH₂)₂ groups. Highly branched polyethylenimines are, e.g., polyethylenimine dendrimers or related molecules with a degree of branching in the range from 0.25 to 0.95, preferably in the range from 0.30 to 0.80 and particularly preferably at least 0.5. The degree of branching can be determined for example by ¹³C-NMR or ¹⁵N-NMR spectroscopy, preferably in D₂O, and is defined as follows:

DB=D+T/D+T+L

with D (dendritic) corresponding to the fraction of tertiary amino groups, L (linear) corresponding to the fraction of secondary amino groups and T (terminal) corresponding to the fraction of primary amino groups.

Within the context of the present invention, branched polyethylenimine units are polyethylenimine units with DB in the range from 0.25 to 0.95, particularly preferably in the range from 0.30 to 0.90% and very particularly preferably at least 0.5. Preferred polyethylenimine units are those that exhibit little or no branching, thus predominantly linear or linear polyethylenimine units.

In the context of the present invention, CH₃-groups are not being considered as branches.

In one embodiment of the present invention polyalkylenimine may have a primary amine value in the range of from 1 to 1000 mg KOH/g, preferably from 10 to 500 mg KOH/g, most preferred from 50 to 300 mg KOH/g. The primary amine value can be determined according to ASTM D2074-07.

In one embodiment of the present invention polyalkylenimine may have a secondary amine value in the range of from 10 to 1000 mg KOH/g, preferably from 50 to 500 mg KOH/g, most preferred from 50 to 500 mg KOH/g. The secondary amine value can be determined according to ASTM D2074-07.

In one embodiment of the present invention polyalkylenimine may have a tertiary amine value in the range of from 1 to 300 mg KOH/g, preferably from 5 to 200 mg KOH/g, most preferred from 10 to 100 mg KOH/g. The tertiary amine value can be determined according to ASTM D2074-07.

In one embodiment of the present invention, the molar share of tertiary N atoms is determined by ¹⁵N-NMR spectroscopy. In cases that tertiary amine value and result according to ¹³C-NMR spectroscopy are inconsistent, the results obtained by ¹³C-NMR spectroscopy will be given preference.

In one embodiment of the present invention, the average molecular weight M_{w} of said polyalkylenimine is in the range of from 250 to 100,000 g/mol, preferably up to 50,000 g/mol and more preferably from 800 up to 25,000 g/mol. The average molecular weight M_{w} of polyalkylenimine may be determined by gel permeation chromatography (GPC) of the intermediate respective polyalkylenimine, with 1.5% by weight aqueous formic acid as eluent and cross-linked polyhydroxyethyl methacrylate as stationary phase.

Said polyalkylenimine may be free or alkoxylated, said alkoxylation being selected from ethoxylation, propoxylation, butoxylation and combinations of at least two of the foregoing. Preference is given to ethylene oxide, 1,2-propylene oxide and mixtures of ethylene oxide and 1,2-propylene oxide. If mixtures of at least two alkylene oxides are applied, they can be reacted step-wise or simultaneously.

In one embodiment of the present invention, an alkoxylated polyalkylenimine bears at least 6 nitrogen atoms per unit.

In one embodiment of the present invention, polyalkylenimine is alkoxylated with 2 to 50 moles of alkylene oxide per NH group, preferably 5 to 30 moles of alkylene oxide per NH group, even more preferred 5 to 25 moles of ethylene oxide or 1,2-propylene oxide or combinations therefrom per NH group. In the context of the present invention, an NH₂ unit is counted as two NH groups. Preferably, all—or almost all—NH groups are alkoxylated, and there are no detectable amounts of NH groups left.

Depending on the manufacture of such alkoxylated polyalkylenimine, the molecular weight distribution may be narrow or broad. For example, the polydispersity Q=M_{w}/Mₙ in the range of from 1 to 3, preferably at least 2, or it may be greater than 3 and up to 20, for example 3.5 to 15 and even more preferred in the range of from 4 to 5.5.

In one embodiment of the present invention, the polydispersity Q of alkoxylated polyalkylenimine is in the range of from 2 to 10.

In one embodiment of the present invention alkoxylated polyalkylenimine is selected from polyethoxylated polyethylenimine, ethoxylated polypropylenimine, ethoxylated α,ω-hexandiamines, ethoxylated and propoxylated polyethylenimine, ethoxylated and propoxylated polypropylenimine, and ethoxylated and poly-propoxylated α,ω-hexandiamines.

In one embodiment of the present invention the average molecular weight Mₙ (number average) of alkoxylated polyethylenimine is in the range of from 2,500 to 1,500,000 g/mol, determined by GPC, preferably up to 500,000 g/mol.

In one embodiment of the present invention, the average alkoxylated polyalkylenimine are selected from ethoxylated α,ω-hexanediamines and ethoxylated and poly-propoxylated α,ω-hexanediamines, each with an average molecular weight Mₙ (number average) in the range of from 800 to 500,000 g/mol, preferably 1,000 to 30,000 g/mol.

Liquid detergent formulations of the invention may comprise one or more corrosion inhibitors. Non-limiting examples of suitable corrosion inhibitors include sodium silicate, triazoles such as benzotriazoles, bisbenzotriazoles, aminotriazoles, alkylaminotriazoles, phenol derivatives such as hydroquinone, pyrocatechol, hydroxyhydroquinone, gallic acid, phloroglucinol and pyrogallol, further polyethylenimine and salts of bismuth or zinc. Corrosion inhibitors may be formulated into liquid detergent formulations of the invention in amounts of 0.1 to 1.5% w/w relative to the overall weight of the liquid detergent composition.

Liquid detergent formulations of the invention may comprise at least one graft copolymer composed of
(a) at least one graft base selected from nonionic monosaccharides, disaccharides, oligosaccharides and polysaccharides,

and side chains obtained by grafting on of
(b) at least one ethylenically unsaturated mono- or dicarboxylic acid and
(c) at least one compound of the general formula (XI),

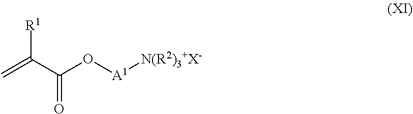

wherein the variables are defined as follows:

R¹ is selected from methyl and hydrogen,

A¹ is selected from C₂-C₄-alkylene,

R² are identical or different and selected from C₁-C₄-alkyl,

X⁻ is selected from halide, mono-C₁-C₄-alkyl sulfate and sulfate.

Liquid detergent formulations of the invention may comprise one or more buffers such as monoethanolamine and N,N,N-triethanolamine.

Liquid detergent formulations of the invention may be adapted in sudsing characteristics for satisfying various purposes. Hand dishwashing detergents usually request stable suds. Automatic dishwasher detergents are usually requested to be low sudsing. Laundry detergents may range from high sudsing through a moderate or intermediate range to low. Low sudsing laundry detergents are usually recommended for front-loading, tumbler-type washers and washer-dryer combinations. Those skilled in the art are familiar with using suds stabilizers or suds suppressors as detergent components in detergent formulations which are suitable for specific applications. Examples of suds stabilizers include but are not limited to alkanolamides and alkylamine oxides. Examples of suds suppressors include but are not limited to alkyl phosphates, silicones and soaps.

Liquid detergent formulations of the invention may comprise one or more fragrances such as benzyl salicylate, 2-(4-tert.-butylphenyl) 2-methylpropional, commercially available as Lilial®, and hexyl cinnamaldehyde.

Liquid detergent formulations of the invention may comprise one or more dyestuffs such as Acid Blue 9, Acid Yellow 3, Acid Yellow 23, Acid Yellow 73, Pigment Yellow 101, Acid Green 1, Solvent Green 7, and Acid Green 25.

Liquid detergent formulations may comprise at least one compound selected from organic solvents, preservatives, viscosity modifiers, and hydrotropes.

In one embodiment of the present invention, liquid detergent formulations comprise amounts of organic solvents are 0.5 to 25% by weight, relative to the total weight of the liquid detergent formulation. Especially when inventive liquid detergent formulations are provided in pouches or the like, 8 to 25% by weight of organic solvent(s) relative to the total weight of the liquid detergent formulation may be comprised. Organic solvents are those disclosed above.

Inventive liquid detergent formulations may comprise one or more preservatives selected from those disclosed above in amounts effective in avoiding microbial contamination of the liquid detergent formulation.

In one embodiment of the present invention, liquid detergent formulations comprise one or more viscosity modifiers. Non-limiting examples of suitable viscosity modifiers include agar-agar, carragene, tragacanth, gum arabic, xanthan gum, alginates, pectins, hydroxyethyl cellulose, hydroxypropyl cellulose, starch, gelatin, locust bean gum, cross-linked poly(meth)acrylates, for example polyacrylic acid cross-linked with bis-(meth)acrylamide, furthermore silicic acid, clay such as but not limited to montmorillonite, zeolite, dextrin, and casein. Viscosity modifiers may be comprised in amounts effective in providing the desired viscosity.

In one embodiment of the present invention, liquid detergent formulations comprise one or more hydrotropes which may be organic solvents such as ethanol, isopropanol, ethylene glycol, 1,2-propylene glycol, and further organic solvents that are water-miscible under normal conditions without limitation. Further examples of suitable hydrotropes are the sodium salts of toluene sulfonic acid, of xylene sulfonic acid, and of cumene sulfonic acid. Hydrotropes may be comprised in amounts that facilitate or enables the dissolution of compounds that exhibit limited solubility in water.

In one embodiment of the present invention, the formulation according to the invention is free from phosphates and polyphosphates, with hydrogenphosphates being subsumed, for example free from trisodiumphosphate, pentasodiumtripolyphosphate and hexasodiummetaphosphate.

In connection with phosphates and polyphosphates, in the context of the present invention, “free from” is to be understood as meaning that the content of phosphate and polyphosphate is in total in the range from 10 ppm to 0.2% by weight, determined by gravimetry.

In one embodiment of the present invention, the formulation according to the invention is free from those heavy metal compounds which do not act as bleach catalysts, in particular from compounds of iron. In connection with heavy metal compounds in the context of the present invention, “free from” is to be understood as meaning that the content of heavy metal compounds which do not act as bleach catalysts is in total in the range from 0 to 100 ppm, preferably 1 to 30 ppm, determined by the Leach method. In the context of the present invention, “heavy metals” are all metals with a specific density of at least 6 g/cm³, with the exception of zinc and bismuth. In particular, heavy metals are precious metals, and also iron, copper, lead, tin, nickel, cadmium and chromium.

In one embodiment, liquid detergent formulations of the invention are free from bleaches, for example free from inorganic peroxide compounds or chlorine bleaches such as sodium hypochlorite, meaning that liquid detergent formulations according to the invention comprise in total 0.01% by weight or less of inorganic peroxide compound and chlorine bleach, relative in each case on total weight of the liquid detergent formulation.

“Detergent formulation” or “cleaning formulation” herein means formulations designated for cleaning soiled material. Cleaning may mean laundering or hard surface cleaning. Soiled material according to the invention includes textiles and/or hard surfaces.

The term “laundering” relates to both household laundering and industrial laundering and means the process of treating textiles with a solution comprising a detergent formulation of the present invention. The laundering process may be carried out by using technical devices such as a household or an industrial washing machine. Alternatively, the laundering process may be done by hand.

The term “textile” means any textile material including yarns (thread made of natural or synthetic fibers used for knitting or weaving), yarn intermediates, fibers, non-woven materials, natural materials, synthetic materials, as well as fabrics (a textile made by weaving, knitting or felting fibers) made of these materials such as garments (any article of clothing made of textile), cloths and other articles.

The term “fibers” includes natural fibers, synthetic fibers, and mixtures thereof. Examples of natural fibers are of plant (such as flax, jute and cotton) or animal origin, comprising proteins like collagen, keratin and fibroin (e.g. silk, sheeps wool, angora, mohair, cashmere). Examples for fibers of synthetic origin are polyurethane fibers such as Spandex® or Lycra®, polyester fibers, polyolefins such as elastofin, or polyamide fibers such as nylon. Fibers may be single fibers or parts of textiles such as knitwear, wovens, or nonwovens.

The term “hard surface cleaning” is defined herein as cleaning of hard surfaces wherein hard surfaces may include any hard surfaces in the household, such as floors, furnishing, walls, sanitary ceramics, glass, metallic surfaces including cutlery or dishes. The term “hard surface cleaning” may therefore may mean “dish washing” which refers to all forms of washing dishes, e.g. by hand or automatic dish wash (ADW). Dish washing includes, but is not limited to, the cleaning of all forms of crockery such as plates, cups, glasses, bowls, all forms of cutlery such as spoons, knives, forks and serving utensils as well as ceramics, plastics such as melamine, metals, china, glass and acrylics.

In one aspect, the invention relates to the providing a liquid detergent formulation comprising at least components (a) and (b) and at least one detergent component, wherein component (b) comprises at least one protease and at least one lipase.

In one embodiment, the invention provides a liquid detergent formulation comprising at least components (a) and (b) and at least one detergent component, wherein component (b) comprises at least one lipase selected from Thermomyces lanuginosa lipase and variants thereof as disclosed above, and at least one protease preferably selected from subtilisin 147 and/or 309 as disclosed in WO 89/06279 and variants thereof having proteolytic activity, subtilisin from Bacillus lentus as disclosed in WO 91/02792 and variants thereof having proteolytic activity, and subtilisin according to SEQ ID NO:22 as described in EP 1921147 and variants thereof having proteolytic activity—all as disclosed above.

In embodiments of the above embodiments, the liquid detergent formulation has increased storage stability when compared to a liquid detergent formulation lacking component (a). Increased storage stability in this context may mean that there is no significant loss in wash performance after storage of the detergent at 37° C. formulation for 1 week [7 days], 2 weeks [14 days], 4 weeks [28 days], 6 weeks [42 days], or 8 weeks [56 days].

No significant loss in wash performance after storage may mean that the detergent has
i. at least 90% wash performance after 4 weeks of storage at 37° C. when compared to the wash performance of the same detergent before storage; and/or
ii. at least 85% wash performance after 6 weeks of storage at 37° C. when compared to the wash performance of the same detergent before storage; and/or
iii. at least 80% wash performance after 8 weeks of storage at 37° C. when compared to the wash performance of the same detergent before storage.

In one embodiment, the liquid detergent formulation comprising at least components (a) and (b) and at least one detergent component has increased storage stability when compared to a liquid detergent formulation lacking component (a), wherein component (b) comprises at least one lipase preferably selected from Thermomyces lanuginosa lipase and variants thereof as disclosed above, and at least one protease as disclosed above, preferably selected from subtilisin 147 and/or 309 as disclosed in WO 89/06279; subtilisin from Bacillus lentus as disclosed in WO 91/02792 and subtilisin according to SEQ ID NO:22 as described in EP 1921147 and variants thereof as disclosed herein. Increased storage stability in one embodiment means that the wash performance of a liquid detergent formulation after 4 to 8 weeks of storage at 37° C. is increased by at least 5%, at least 6%, at least 7%, at least 8%, at least 9%, or at least 10% when compared to a liquid detergent formulation lacking component (a) stored for the same time at the same temperature. Increased storage stability may mean that the wash performance of a liquid detergent formulation after 8 weeks of storage at 37° C. is increased by at least 5%, at least 6%, at least 7%, at least 8%, at least 9%, or at least 10% when compared to a liquid detergent formulation lacking component (a) stored for the same time at the same temperature.

In one aspect, the invention relates to the providing a liquid detergent formulation comprising at least components (a) and (b) and at least one detergent component, wherein component (b) comprises at least one protease and at least one amylase.

In one embodiment, the invention provides a liquid detergent formulation comprising at least components (a) and (b) and at least one detergent component, wherein component (b) comprises at least one preferably selected from subtilisin 147 and/or 309 as disclosed in WO 89/06279 and variants thereof having proteolytic activity, subtilisin from Bacillus lentus as disclosed in WO 91/02792 and variants thereof having proteolytic activity, and subtilisin according to SEQ ID NO:22 as described in EP 1921147 and variants thereof having proteolytic activity, and at least one amylase preferably selected from amylases from Bacillus sp. 707 or variants thereof having amylolytic activity and amylases from Bacillus halmapalus or variants thereof having amylolytic activity, more preferably selected from amylases according to SEQ ID NO: 1 and 2 of WO 2013/001078 or variants thereof having amylolytic activity—all as disclosed above. In one aspect, the invention relates to the use of component (a) to stabilize component (b) within a liquid detergent formulation, wherein component (b) comprises at least one protease as disclosed above, preferably selected from subtilisin 147 and/or 309 as disclosed in WO 89/06279 or variants thereof having proteolytic activity, subtilisin from Bacillus lentus as disclosed in WO 91/02792 or variants thereof having proteolytic activity, and subtilisin according to SEQ ID NO:22 as described in EP 1921147 or variants thereof having proteolytic activity all as disclosed herein.

In one embodiment, the invention relates to the use of component (a) to stabilize component (b) within a liquid detergent formulation, wherein component (b) comprises at least one protease as disclosed above, preferably selected from subtilisin 147 and/or 309 as disclosed in WO 89/06279 or variants thereof having proteolytic activity, subtilisin from Bacillus lentus as disclosed in WO 91/02792 or variants thereof having proteolytic activity, and subtilisin according to SEQ ID NO:22 as described in EP 1921147 or variants thereof having proteolytic activity, and at least one lipase preferably selected from Thermomyces lanuginosa lipase and variants thereof as disclosed above—all enzymes as disclosed herein.

In one embodiment, the invention relates to the use of component (a) to stabilize component (b) within a liquid detergent formulation, wherein component (b) comprises at least one protease as disclosed above, preferably selected from subtilisin 147 and/or 309 as disclosed in WO 89/06279 or variants thereof having proteolytic activity, subtilisin from Bacillus lentus as disclosed in WO 91/02792 or variants thereof having proteolytic activity, and subtilisin according to SEQ ID NO:22 as described in EP 1921147 or variants thereof having proteolytic activity, and at least one amylase preferably selected from amylases from Bacillus sp. 707 or variants thereof having amylolytic activity and amylases from Bacillus halmapalus or variants thereof having amylolytic activity, more preferably selected from amylases according to SEQ ID NO: 1 and 2 of WO 2013/001078 or variants thereof having amylolytic activity—all enzymes as disclosed herein.

Stabilized component (b) in this context means that the wash performance of a liquid detergent formulation comprising component (b) after 4 to 8 weeks of storage at 37° C. is increased by at least 5%, at least 6%, at least 7%, at least 8%, at least 9%, or at least 10% when compared to a liquid detergent formulation lacking component (a) stored for the same time at the same temperature. Stabilized component (b) may mean that the wash performance of a liquid detergent formulation comprising component (b) after 8 weeks of storage at 37° C. is increased by at least 5%, at least 6%, at least 7%, at least 8%, at least 9%, or at least 10% when compared to a liquid detergent formulation lacking component (a) stored for the same time at the same temperature.

In one aspect, the invention relates to the use of component (a) to reduce loss of enzymatic activity during storage, preferably at 37° C. for 21, 28 and/or 35 days, of component (b) within a liquid detergent formulation, wherein component (b) comprises at least one protease, preferably selected from subtilisin 147 and/or 309 as disclosed in WO 89/06279 and variants thereof having proteolytic activity, subtilisin from Bacillus lentus as disclosed in WO 91/02792 and variants thereof having proteolytic activity, and subtilisin according to SEQ ID NO:22 as described in EP 1921147 and variants thereof having proteolytic activity, and at least one lipase preferably selected from Thermomyces lanuginosa lipase and variants thereof—all enzymes as disclosed above.

In one aspect, the invention relates to the use of component (a) to reduce loss of enzymatic activity during storage, preferably at 37° C. for 21, 28 and/or 35 days, of component (b) within a liquid detergent formulation, wherein component (b) comprises at least one protease, preferably selected from subtilisin 147 and/or 309 as disclosed in WO 89/06279 and variants thereof having proteolytic activity, subtilisin from Bacillus lentus as disclosed in WO 91/02792 and variants thereof having proteolytic activity, and subtilisin according to SEQ ID NO:22 as described in EP 1921147 and variants thereof having proteolytic activity, and at least one amylase preferably selected from amylases from Bacillus sp. 707 or variants thereof having amylolytic activity and amylases from Bacillus halmapalus or variants thereof having amylolytic activity, more preferably selected from amylases according to SEQ ID NO: 1 and 2 of WO 2013/001078 or variants thereof having amylolytic activity—all enzymes as disclosed above.

In one aspect, the invention relates to a method to increase storage stability of a liquid detergent formulation comprising at least one protease, by adding at least one compound according to formula (I) to the detergent formulation:

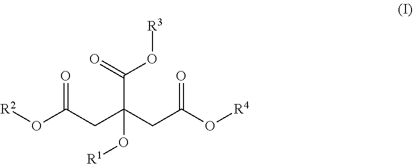

wherein the variables of formula (I) are as follows:

R¹ is selected from H and C₁-C₁₀ alkylcarbonyl, wherein alkyl may be linear or branched and may bear one or more hydroxyl groups;

R², R³, R⁴ are independently from each other selected from H, linear C₁-C₈ alkyl, and branched C₃-C₈ alkyl, C₆-C₁₀-aryl, non-substituted or substituted with one or more carboxylate or hydroxyl groups, and C₆-C₁₀-aryl-alkyl, wherein alkyl of the latter is selected from linear C₁-C₈ alkyl or branched C₃-C₈ alkyl, wherein at least one of R², R³, and R⁴ is not H.

In one embodiment, storage stability of said liquid detergent formulation is increased during storage at 37° C. for 21, 28 and/or 35 days when compared to a liquid detergent formulation lacking the compound according to formula (I) stored under the same conditions. Increased storage stability within this invention may mean that the increase in enzyme stability in the presence of component (a) is at least 5%, at least 10%, at least 15%, at least 20%, at least 25%, at least 30%, at least 40%, at least 50%, least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99.5%, when compared to the enzymatic activity in the absence of component (a).

In one embodiment, said liquid detergent formulation comprises at least one protease selected from the group of subtilisin type proteases (EC 3.4.21.62), and wherein the liquid detergent formulation optionally comprises at least one lipase, preferably selected from Thermomyces lanuginosa lipase, wherein
(a) at least one protease is preferably selected from subtilisin 147 and/or 309 as disclosed in WO 89/06279 or variants thereof having proteolytic activity, subtilisin from Bacillus lentus as disclosed in WO 91/02792 or variants thereof having proteolytic activity, and subtilisin according to SEQ ID NO:22 as described in EP 1921147 or variants thereof having proteolytic activity—all as disclosed herein, and
(b) at least one lipase is preferably selected from Thermomyces lanuginosa lipase and variants thereof as disclosed above.

In one embodiment, said liquid detergent formulation comprises at least one protease selected from the group of subtilisin type proteases (EC 3.4.21.62), and wherein the liquid detergent formulation optionally comprises at least one amylase, wherein
(a) at least one protease is preferably selected from subtilisin 147 and/or 309 as disclosed in WO 89/06279 or variants thereof having proteolytic activity, subtilisin from Bacillus lentus as disclosed in WO 91/02792 or variants thereof having proteolytic activity, and subtilisin according to SEQ ID NO:22 as described in EP 1921147 or variants thereof having proteolytic activity—all as disclosed herein, and
(b) at least one amylase is preferably selected from amylases from Bacillus sp. 707 or variants thereof having amylolytic activity and amylases from Bacillus halmapalus or variants thereof having amylolytic activity, more preferably selected from amylases according to SEQ ID NO: 1 and 2 of WO 2013/001078 or variants thereof having amylolytic activity—as disclosed herein.

### Further Use

The invention relates to a method for removing stains comprising the steps of contacting a stain with a detergent formulation of the invention comprising components (a) and (b) and one or more detergent components. In one embodiment, the method for removing stains includes steps performed by an automatic device such as a laundry machine or an automatic dishwasher.

In one embodiment, the detergent formulation comprises the enzyme preparation of the invention.

In one aspect, the method relates to the removal of stains comprising fat. Fats can be sub-classified as fat, grease or oil depending on the melting temperature. Oil is usually liquid at room temperature. Grease has a higher viscosity than oil at room temperature and may be called pasty. In one embodiment, removing of stains comprising fat may be done at cleaning temperatures ≤40° C., at cleaning temperatures ≤30° C., at cleaning temperatures ≤25° C., or at cleaning temperatures ≤20° C.

In one aspect, the invention relates to a method for removing stains comprising fatty compounds having a melting temperature below the cleaning temperature. In one embodiment, the stain to be removed from a textile comprises fatty compounds having a melting temperature of >30° C., and the removal is done at a cleaning temperature of temperature ≤30° C.

In one embodiment, the invention relates to a method for removing stains comprising fatty compounds having a melting temperature >30° C. at a cleaning temperature of temperature ≤30° C., wherein the method comprises the steps of contacting the stain with a detergent formulation of the invention comprising components (a) and (b) and one or more detergent components. Components (a) and (b) are those as disclosed above. Component (b), in one embodiment comprises at least one lipase preferably selected from Thermomyces lanuginosa lipase and variants thereof and at least one protease, preferably selected from subtilisin 147 and/or 309 as disclosed in WO 89/06279 and variants thereof having proteolytic activity, subtilisin from Bacillus lentus as disclosed in WO 91/02792 and variants thereof having proteolytic activity, and subtilisin according to SEQ ID NO:22 as described in EP 1921147 and variants thereof having proteolytic activity—all as disclosed above.

In one aspect, the method relates to the removal of stains comprising starch. In one embodiment, removing of stains comprising starch may be done at cleaning temperatures ≤40° C., at cleaning temperatures ≤30° C., at cleaning temperatures ≤25° C., or at cleaning temperatures ≤20° C.

In one embodiment, the invention relates to a method for removing stains comprising starch at a cleaning temperature of temperature ≤30° C., wherein the method comprises the steps of contacting the stain with a detergent formulation of the invention comprising components (a) and (b) and one or more detergent components. Components (a) and (b) are those as disclosed above. Component (b), in one embodiment comprises at least one protease, preferably selected from subtilisin 147 and/or 309 as disclosed in WO 89/06279 and variants thereof having proteolytic activity, subtilisin from Bacillus lentus as disclosed in WO 91/02792 and variants thereof having proteolytic activity, and subtilisin according to SEQ ID NO:22 as described in EP 1921147 and variants thereof having proteolytic activity, and at least one amylase preferably selected from amylases from Bacillus sp. 707 or variants thereof having amylolytic activity and amylases from Bacillus halmapalus or variants thereof having amylolytic activity, more preferably selected from amylases according to SEQ ID NO: 1 and 2 of WO 2013/001078 or variants thereof having amylolytic activity—all enzymes as disclosed above.

### EXAMPLES

The invention will be further illustrated by working examples.

General remarks: percentages are weight percent unless specifically noted otherwise.

### I. Tested Compounds

### A) Compounds According to Formula (I)—(Component (a)):

A.1 Triethylcitrate—purchased from Sigma Aldrich

A.2 Tripropylcitrate—purchased from Sigma Aldrich

A.3 Tributylcitrate—purchased from Sigma Aldrich

A.4 Acetyltributylcitrate—purchased from Sigma Aldrich

A.5 Acetyltriethylcitrate—purchased from Sigma Aldrich

A.6 Monoethylcitrate—purchased from Sigma Aldrich

A.7 Diethylcitrate
Synthesis of as described in: Journal of Chemical & Engineering Data 2018, DOI: 10.1021/acs.jced.7b01060, C. Berdugo, A. Suaza, M. Santaella, O. Sanchez

A.8 Tribenzylcitrate
Synthesis as described in WO2007/14471 A1, 2007; Location in patent: Page/Page column 19; 27-28

A.9 Trisalicylcitrate
Synthesis as described in WO2007/14471 A1, 2007; Location in patent: Page/Page column 19; 27-28

### B) Comparative Compounds:

B.1: citric acid—purchased from Sigma Aldrich

B.2: citric acid trisodium salt—purchased from Sigma Aldrich

B.3: diethyloxalate—purchased from Sigma Aldrich

B.4: glyceroltriacetate (triacetine)—purchased from Sigma Aldrich

### II. Protease Stability

The storage stability of protease was assessed at 37° C.

Base test formulations were manufactured by making base formulations I to V by mixing the components according to Table 1.

Protease used: Savinase® 16.0L (CAS-No. 9014-01-1, EC-No. 232-752-2) was purchased from Sigma-Aldrich.

The respective component (a) or comparative compound was added, if applicable, to the respective base formulation in amounts as indicated in Table 1.

Protease (component (b)) was added, to the respective base formulation in amounts as indicated in Table 1. The amount of protease as provided in Table 1 refers to active protein.

Water was added to accomplish the balance to 100.

TABLE 1
liquid formulations

| | wt % in formulation | | | | |
|---|---|---|---|---|---|
| Ingredients | I. | II. | III. | IV. | V. |
| Base formulation: | | | | | |
| (Comp. 1) | 15 | 8 | — | 6 | 6 |
| (Comp. 2) | — | 6 | 8 | 8 | 8 |
| (Comp. 3) | 6 | 4 | — | 4 | 4 |
| (Comp. 4) | 2 | — | — | 2 | — |
| (Comp. 5) | — | 4 | 8 | 4 | 4 |
| (Comp. 6) | — | 2.5 | — | — | 2.5 |
| Sorbitol | 3 | — | — | 2 | — |
| PEI-EO20 | 3 | 5 | 3 | 5 | 5 |
| Propyleneglycol | — | 4 | — | 2 | 4 |
| Glycerol | — | — | 6 | — | — |
| Ca-formiate | 1 | — | 1 | — | — |
| Additives: | | | | | |
| Savinase 16.0 L | 0.7 | 0.7 | 0.7 | 0.7 | 0.7 |
| component (a)** | 2.5 | 2.5 | 2.0 | 2.0 | 2.0 |

| | |
|---|---|
| balance | Water to 100 |

(Comp. 1): n-C₁₈-alkyl-(OCH₂CH₂)₂₅—OH
(Comp. 2): C₁₀-C₁₈-alkylpolygycoside blend
(Comp. 3): Sodium C₁₀-C₁₂-alkyl benzenesulfonate
(Comp. 4): Sodium cumenesulfonate
(Comp. 5): Sodium laurethsulfate - n-C₁₂H₂₅—O—(CH₂CH₂O)₃—SO₃Na
(Comp. 6): n-C₁₂H₂₅(CH₃)₂N→O
**for comparative tests without inventive compounds those were replaced by the same amount of water.

Savinase activity at certain points in time as indicated in Table 2 was be determined by employing Succinyl-Ala-Ala-Pro-Phe-p-nitroanilide (Suc-AAPF-pNA, short AAPF) as substrate. pNA is cleaved from the substrate molecule by proteolytic cleavage, resulting in release of yellow color of free pNA which was determined by measuring OD₄₀₅. Measurements were done at 20° C.

Table 2 displays protease activity measured in liquid formulations after storage for 1 to 30 days at 37° C. The proteolytic activity values provided in Table 3 were calculated referring to the 100% value determined in the reference formulation at the time 0.

The nomenclature of formulations is as follows: the Roman number before the full stop characterizes the base formulation, the Arabian number the type of compound (A. # compound according to invention (component (a)); (B. #) comparative compound).

TABLE 2
protease activity in the course of time of storage at 37° C.

| Formulation identifier | | | | | | | | |
|---|---|---|---|---|---|---|---|---|
| Base | com- | | | | | | | |
| formulation | pound | T0 | 3 d | 7 d | 14 d | 21 d | 28 d | 35 d |

| | | | | | | | |
|---|---|---|---|---|---|---|---|
| I. 0 | 100 | 93 | 87 | 78 | 69 | 64 | 59 |
| I. (A.1) | 100 | 96 | 93 | 89 | 83 | 79 | 72 |
| I. (A.2) | 101 | 100 | 94 | 91 | 85 | 80 | 76 |
| I. (A.4) | 102 | 100 | 96 | 92 | 87 | 83 | 79 |
| I. (A.5) | 98 | 96 | 94 | 90 | 85 | 82 | 78 |
| I. (A.7) | 100 | 95 | 91 | 85 | 81 | 76 | 69 |
| I. (B.1) | 98 | 94 | 86 | 76 | 67 | 62 | 57 |
| I. (B.2) | 97 | 95 | 84 | 68 | 64 | 59 | 55 |
| I. (B.3) | 99 | 90 | 86 | 70 | 65 | 62 | 58 |
| I. (B.4) | 100 | 91 | 84 | 70 | 66 | 61 | 56 |
| II. 0 | 98 | 93 | 89 | 74 | 69 | 64 | 59 |
| II. (A.1) | 100 | 100 | 96 | 93 | 89 | 83 | 78 |
| II. (A.5) | 102 | 101 | 100 | 94 | 90 | 85 | 81 |
| II. (A.6) | 101 | 102 | 100 | 95 | 92 | 87 | 83 |
| II. (A.7) | 98 | 98 | 96 | 94 | 90 | 87 | 83 |
| II. (A.8) | 100 | 94 | 91 | 88 | 85 | 83 | 77 |
| II. (A.9) | 98 | 96 | 92 | 87 | 84 | 80 | 76 |
| II. (B.2) | 98 | 95 | 82 | 75 | 66 | 63 | 60 |
| II. (B.4) | 97 | 87 | 80 | 73 | 58 | 57 | 54 |
| III. 0 | 96 | 93 | 85 | 75 | 64 | 60 | 55 |
| III. (A.1) | 100 | 98 | 94 | 91 | 87 | 84 | 81 |
| III. (A.2) | 102 | 100 | 100 | 96 | 91 | 88 | 84 |
| III. (A.5) | 99 | 97 | 95 | 91 | 86 | 81 | 77 |
| III. (A.6) | 99 | 96 | 92 | 86 | 82 | 79 | 76 |
| III. (A.7) | 100 | 96 | 93 | 84 | 83 | 76 | 72 |
| III. (A.9) | 102 | 98 | 97 | 95 | 91 | 87 | 83 |
| III. (B.1) | 100 | 92 | 84 | 75 | 65 | 60 | 53 |
| III. (B.2) | 101 | 93 | 83 | 73 | 62 | 57 | 50 |
| III. (B.3) | 98 | 92 | 85 | 74 | 60 | 55 | 51 |
| IV. 0 | 96 | 90 | 85 | 77 | 68 | 60 | 55 |
| IV. (A.1) | 98 | 96 | 93 | 88 | 82 | 79 | 74 |
| IV. (A.3) | 99 | 100 | 96 | 91 | 84 | 81 | 77 |
| IV. (B.2) | 98 | 91 | 86 | 70 | 64 | 58 | 52 |
| V. 0 | 98 | 93 | 89 | 74 | 68 | 62 | 57 |
| V. (A.1) | 97 | 94 | 90 | 86 | 83 | 79 | 75 |
| V. (A.3) | 101 | 98 | 93 | 88 | 85 | 80 | 74 |
| V. (A.8) | 96 | 96 | 92 | 86 | 84 | 80 | 73 |
| V. (B.1) | 97 | 93 | 86 | 75 | 65 | 57 | 50 |

### III. Textile Cleaning Tests

The detergent performance of formulations in cleaning two types of test fabrics was carried out. Testing cloth samples comprised a complex soil comprising proteinaceous and fatty components due to CFT process as well as test cloth samples comprised a fatty/particulate type of soil.

The test was performed as follows: a multi stain monitor comprising 8 standardized soiled fabric patches, each of 2.5×2.5 cm size and stitched on two sides to a polyester carrier was washed together in a launder-O-meter with 2.5 g of cotton fabric and 5 g/L of the liquid test laundry detergent, Table 3.

The conditions were as follows: Device: Launder-O-Meter from SDL Atlas, Rock Hill, USA. Washing liquor: 250 ml, washing time: 60 minutes, washing temperature: 30° C. Water hardness: 2.5 mmol/L; Ca:Mg:HCO₃ 4:1:8

Fabric to liquor ratio 1:12 After the wash cycle, the multi stain monitors were rinsed in water, followed by drying at ambient temperature over a time period of 14 hours.

The following pre-soiled test fabrics were used:

CFT C-S-10: butter on cotton

CFT C-S-62: lard, colored on cotton

CFT C-S-68: chocolate ice-cream on cotton

EMPA 112: cocoa on cotton

EMPA 141/1: lipstick on cotton

EMPA 125: monitor for surfactant

wfk20D: pigment and sebum-type fat on polyester/cotton mixed fabric

CFT C-S-70: chocolate mousse

wfk=wfk test fabrics GmbH, Krefeld

EMPA=Swiss Federal Institute of Materials Testing

CFT=Center for Test Material B.V.

The total level of cleaning was evaluated using color measurements. Reflectance values of the stains on the monitors were measured using a sphere reflectance spectrometer (SF 500 type from Datacolor, USA, wavelength range 360-700 nm, optical geometry d/8°) with a UV cutoff filter at 460 nm. In this case, with the aid of the CIE-Lab color space classification, the brightness L*, the value a* on the red-green color axis and the b* value on the yellow-blue color axis, were measured before and after washing and averaged for the 8 stains of the monitor. The change of the color value (ΔE) value, defined and calculated automatically by the evaluation color tools on the following equation:

ΔE*_{ab}=\sqrt{ΔL*²+Δa*²+Δb*²}

[L* brightness, a* color value on red-green axis, b* color value on blue-yellow axis]

ΔE is a measure of the achieved cleaning effect. All measurements were repeated six times to yield an average number. Note that higher A E values show better cleaning. A difference of 1 unit can be detected by a skilled person. A non-expert can detect 2 units easily. The results are shown in Table 4.

R_{w}=washed soil reflectance

Rₒ=unsoiled reflectance

The detergency was calculated as: A total of 6 replications of each cloth were run during this study; a statistical confidence level of 90-95% was calculated.

Test formulations were manufactured by making formulations VI to X by mixing the components according to Table 3.

The respective component (a) or comparative compound was added, if applicable, to the respective base formulation in amounts provided in Table 3.

Lipolase® 100L was added, if applicable, to the respective base formulation in amounts provided in Table 3.

Savinase® 16.0L was added, if applicable, to the respective base formulation in amounts provided in Table 3.

Water was added to accomplish the balance to 100.

TABLE 3
liquid laundry formulations

| | Wt-% in formulation | | | | |
|---|---|---|---|---|---|
| Ingredients | VI. | VII. | VIII. | IX. | X. |
| Base formulation: | | | | | |
| (Comp. 1) | 8 | 8 | 8 | 8 | 8 |
| (Comp. 2) | 6 | 6 | 6 | 6 | 6 |
| (Comp. 3) | 4 | 4 | 4 | 4 | 4 |
| (Comp. 4) | 4 | 4 | 4 | 4 | 4 |
| (Comp. 5) | 2.5 | 2.5 | 2.5 | 2.5 | 2.5 |
| PEI-EO20 | 5 | 5 | 5 | 5 | 5 |
| Propyleneglycol | 4 | 4 | 4 | 4 | 4 |
| Additives: | | | | | |
| Savinase 16.0 L | — | — | — | 0.7 | 0.7 |
| Lipolase | — | — | 0.2 | 0.2 | 0.2 |
| component (a)** | — | 2.5 | 2.5 | — | 2.5 |

| | |
|---|---|
| balance | Water to 100 |

(Comp. 1): n-C₁₈-alkyl-(OCH₂CH₂)₂₅—OH
(Comp. 2): C₁₀-C₁₈-alkylpolygycoside blend
(Comp. 3): Sodium C₁₀-C₁₂-alkyl benzenesulfonate
(Comp. 4): Sodium laurethsulfate - n-C₁₂H₂₅—O—(CH₂CH₂O)₃—SO₃Na
(Comp. 5): n-C₁₂H₂₅(CH₃)₂N→O
**for comparative tests without inventive compounds those were replaced by the same amount of water.

The launder-O-meter tests were executed with freshly prepared formulations and with formulations stored at 37° C. during a 2-month storage (1 week [7 days], 2 weeks [14 days], 4 weeks [28 days], 6 weeks [42 days], 8 weeks [56 days]). As an approximation one week at 37° C. is equivalent to 3½ weeks at 20° C.

TABLE 4
Results of launder-O-meter tests: sum of ΔE of
the above mentioned multi-stain monitor

| Formulation identifier | | | | | | | |
|---|---|---|---|---|---|---|---|
| Base | | | ΔE | ΔE | ΔE | ΔE | ΔE |
| formulation | compound | ΔE T0 | 1 week | 2 weeks | 4 weeks | 6 weeks | 8 weeks |
| VI. | — | 152 | 154 | 153 | 151 | 153 | 153 |
| VII. | A.1 | 154 | 153 | 154 | 152 | 152 | 153 |
| VII. | A.2 | 152 | 152 | 154 | 152 | 152 | 153 |
| VII. | A.5 | 154 | 155 | 153 | 153 | 152 | 153 |
| VII. | A.8 | 153 | 153 | 152 | 152 | 152 | 151 |
| VIII. | 0 | 183 | 184 | 181 | 179 | 179 | 175 |
| VIII. | A.3 | 185 | 185 | 181 | 178 | 176 | 173 |
| VIII. | A.4 | 185 | 185 | 183 | 181 | 182 | 181 |
| VIII. | A.7 | 182 | 179 | 179 | 175 | 173 | 170 |
| IX. | — | 187 | 183 | 176 | 172 | 165 | 159 |
| X. | A.1 | 191 | 188 | 187 | 184 | 184 | 180 |
| X. | A.2 | 189 | 187 | 184 | 182 | 182 | 177 |
| X. | A.5 | 190 | 187 | 187 | 183 | 180 | 180 |
| X. | A.8 | 191 | 189 | 185 | 186 | 182 | 176 |
| X. | B.1 | 190 | 186 | 180 | 175 | 168 | 160 |
| X. | B.3 | 190 | 187 | 182 | 177 | 169 | 162 |
| X. | B.4 | 188 | 185 | 181 | 173 | 166 | 159 |

1. An enzyme preparation comprising
component (a): at least one compound according to general formula (I) 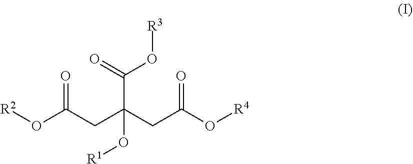
wherein the variables in formula (I) are defined as follows:
R¹ is H;
R², R³, R⁴ are independently from each other selected from the group consisting of H, linear C₁-C₈ alkyl, and branched C₃-C₈ alkyl, C₆-C₁₀-aryl, non-substituted or substituted with one or more carboxylate or hydroxyl groups, and C₆-C₁₀-aryl-alkyl, wherein an alkyl of the C₆-C₁₀-aryl-alkyl is selected from the group consisting of linear C₁-C₈ alkyl and branched C₃-C₈ alkyl, wherein at least one of R², R³, and R⁴ is not H;
component (b): at least one enzyme selected from the group consisting of proteases,
and optionally
component (c): a compound selected from the group consisting of at least one solvent, at least one enzyme stabilizer different from component (a), and at least one compound stabilizing the enzyme preparation.

2. The enzyme preparation according to claim 1, wherein said enzyme preparation comprises component (a) in amounts in a range of 0.1 to 30% by weight relative to a total weight of the enzyme preparation.

3. The enzyme preparation according to claim 1, characterized in that the at least one enzyme comprised in component (b) is stabilized when compared to an enzyme preparation lacking component (a).

4. A process for making a stable enzyme preparation, said process comprising the steps of mixing at least
component (a): at least one compound according to general formula (I) 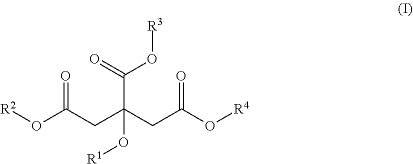
wherein the variables in formula (I) are defined as follows:
R¹ is H;
R², R³, R⁴ are independently from each other selected from the group consisting of H, linear C₁-C₈ alkyl, and branched C₃-C₈ alkyl, C₆-C₁₀-aryl, non-substituted or substituted with one or more carboxylate or hydroxyl groups, and C₆-C₁₀-aryl-alkyl, wherein an alkyl of the C₆-C₁₀-aryl-alkyl is selected from the group consisting of linear C₁-C₈ alkyl and branched C₃-C₈ alkyl, wherein at least one of R², R³, and R⁴ is not H,
component (b): at least one enzyme selected from the group consisting of proteases,
and optionally
component (c): a compound selected from the group consisting of at least one solvent, at least one enzyme stabilizer different from component (a), and at least one compound stabilizing the enzyme preparation.

5. A method of reducing loss of proteolytic activity of at least one protease comprised in a liquid enzyme preparation during storage, the method comprising the step of adding to the liquid enzyme preparation at least one compound according to formula (I): 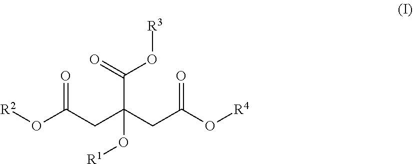
wherein the variables in formula (I) are defined as follows:
R¹ is H;
R², R³, R⁴ are independently from each other selected from the group consisting of H, linear C₁-C₈ alkyl, and branched C₃-C₈ alkyl, C₆-C₁₀-aryl, non-substituted or substituted with one or more carboxylate or hydroxyl groups, and C₆-C₁₀-aryl-alkyl, wherein an alkyl of the C₆-C₁₀-aryl-alkyl is selected from the group consisting of linear C₁-C₈ alkyl and branched C₃-C₈ alkyl, wherein at least one of R², R³, and R⁴ is not H.

6. A method of using a compound according to formula (I): 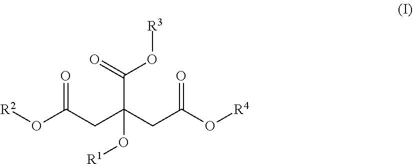
wherein the variables in formula (I) are defined as follows:
R¹ is H;
R², R³, R⁴ are independently from each other selected from the group consisting of H, linear C₁-C₈ alkyl, and branched C₃-C₈ alkyl, C₆-C₁₀-aryl, non-substituted or substituted with one or more carboxylate or hydroxyl groups, and C₆-C₁-aryl-alkyl, wherein an alkyl of the C₆-C₁₀-aryl-alkyl is selected from linear C₁-C₈ alkyl and branched C₃-C₈ alkyl, wherein at least one of R², R³, and R⁴ is not H,
the method comprising using the compound according to formula (I) as an additive for at least one protease, wherein the compound according to formula (I) and the protease are solid and wherein proteolytic activity of the protease is stabilized when the compound according to formula (I) and the protease are contacted with at least one solvent [component (c)].

7. A method of using the enzyme preparation of claim 1 to formulate detergent formulations, the method comprising mixing the enzyme preparation in one or more steps with one or more detergent components.

8. A detergent formulation comprising the enzyme preparation of claim 1 and at least one detergent component.

9. A method of preparing a detergent formulation comprising the steps of mixing at least
component (a): at least one compound according to general formula (I) 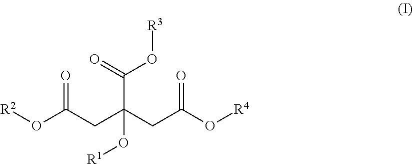
wherein the variables of formula (i) are as follows:
R¹ is H;
R², R³, R⁴ are independently from each other selected from the group consisting of H, linear C₁-C₈ alkyl, and branched C₃-C₈ alkyl, C₆-C₁₀-aryl, non-substituted or substituted with one or more carboxylate or hydroxyl groups, and C₆-C₁₀-aryl-alkyl, wherein an alkyl of the C₆-C₁₀-aryl-alkyl is selected from the group consisting of linear C₁-C₈ alkyl and branched C₃-C₈ alkyl, wherein at least one of R², R³, and R⁴ is not H,
component (b): at least one enzyme selected from the group consisting of proteases,
and at least one detergent component in effective amounts.

10. A method of preparing a detergent formulation, comprising the steps of mixing the enzyme preparation of claim 1 and at least one detergent component in effective amounts.

11. A method for removing stains, comprising the step of contacting at least one stain with the detergent formulation according to claim 8, wherein component (b) of said detergent formulation comprises at least one protease, and optionally further comprises at least one lipase.

12. The method according to claim 11, wherein the stain is to be removed from a textile and the stain comprises fatty compounds having a melting temperature of >30° C., and the removal is done at a cleaning temperature of ≤30° C.

13. A method to increase storage stability of a liquid detergent formulation comprising at least one protease, the method comprising adding at least one compound according to formula (I) to the detergent formulation: 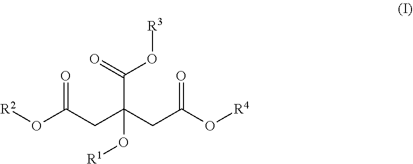
wherein the variables of formula (I) are as follows:
R¹ is H;
R², R³, R⁴ are independently from each other selected from the group consisting of H, linear C₁-C₈ alkyl, and branched C₃-C₈ alkyl, C₆-C₁₀-aryl, non-substituted or substituted with one or more carboxylate or hydroxyl groups, and C₆-C₁₀-aryl-alkyl, wherein an alkyl of the C₆-C₁₀-aryl-alkyl is selected from the group consisting of linear C₁-C₈ alkyl and branched C₃-C₈ alkyl, wherein at least one of R², R³, and R⁴ is not H.

14. The method according to claim 13, wherein the detergent formulation is stored at 37° C. for at least 20 days.

15. The method according to claim 13, wherein the protease is selected from the group consisting of subtilisin type proteases (EC 3.4.21.62), and wherein the liquid detergent formulation optionally comprises at least one lipase.

16. The enzyme preparation according to claim 1, wherein the at least one enzyme is selected from the group consisting of serine endopeptidases (EC 3.4.21) and subtilisin type proteases (EC 3.4.21.62).

17. The process according to claim 4, wherein the at least one enzyme is selected from the group consisting of serine endopeptidases (EC 3.4.21) and subtilisin type proteases (EC 3.4.21.62).

18. The method according to claim 7, wherein the detergent formulations are liquid detergent formulations.

19. The method according to claim 9, wherein the at least one enzyme is selected from the group consisting of serine endopeptidases (EC 3.4.21) and subtilisin type proteases (EC 3.4.21.62).

20. The method according to claim 15, wherein the lipase is selected from the group consisting of Thermomyces lanuginosa lipase and variants thereof.
